(19) United States
(12) Patent Application Publication
(10) Pub. No.: US 2024/0317885 A1
(43) Pub. Date: Sep. 26, 2024

(54) ANTI-TAX INTERACTING PROTEIN-1 (TIP1) BINDING MOLECULES

(71) Applicants: Medical Guidance Systems, LLC, St. Louis, MO (US); Washington University, St. Louis, MO (US)

(72) Inventors: Dennis Hallahan, St. Louis, MO (US); Abhay Kumar Singh, St. Louis, MO (US); Vaishali Kapoor, St. Louis, MO (US); Sapna Deore, St. Louis, MO (US)

(21) Appl. No.: 18/579,108

(22) PCT Filed: Jul. 14, 2022
(86) PCT No.: PCT/US2022/037060

§ 371 Date: Jan. 12, 2024

Related U.S. Application Data
(60) Provisional application No. 63/222,795, filed on Jul. 16, 2021.

(51) Int. Cl. C07K 16/30; A61K 39/00; A61P 35/00

(52) U.S. Cl. CPC C07K 16/30; A61P 35/00; A61K 2039/505; C07K 2317/24; C07K 2317/33; C07K 2317/622; C07K 2317/92

(57) ABSTRACT

Disclosed herein is a novel class of isolated binding molecules including monoclonal antibodies that target Tax interacting protein-I (TIP1) and in certain embodiments can inhibit cancer cell migration, metastasis, and/or viability. Certain embodiments provide for the prevention, treatment, and management of tumors and/or cancer.

### CROSS-REFERENCE TO RELATED APPLICATIONS

This application claims the benefit of U.S. Provisional Application No. 63/222,795, filed Jul. 16, 2021, which is incorporated herein by reference in its entirety.

### STATEMENT REGARDING FEDERALLY-SPONSORED RESEARCH AND DEVELOPMENT

This invention was made with government support under R44CA210687 (Humanization, Production, and PK/PD Characterization of anti-TIP1 antibody against Non-Small Cell Lung Cancer) awarded by the National Institutes of Health (NIH). The government has certain rights in the invention.

### BACKGROUND

### Field of the Invention

This disclosure is drawn to a novel class of isolated binding molecules including monoclonal antibodies that target Tax interacting protein-1 (TIP1) and methods of making the same. Further provided are methods for the prevention, treatment, and management of tumors and/or cancer.

### Background Art

Delivery of radiation-sensitizers to radiation-inducible antigens by the use of antibodies has previously been described (Lewis C D, et al. Clin Cancer Res. 2021:27(11): 3224-33). There are many examples of therapeutic uses of antibodies to treat cancer that include unconjugated (naked) therapeutic antibodies. These antibodies block the function of the target proteins like EGFR and PD-L1 on the surface of cancer cells (Gray J E, et al. Journal of thoracic oncology: official publication of the International Association for the Study of Lung Cancer. 2020:15(2):288-93: Bonner J A, et al. The Lancet Oncology. 2010:11(1):21⁻⁸). Secondly, antibody drug conjugates consist of chemotherapeutic agents covalently linked to antibodies. The cytotoxic agents are released following endocytosis of the antibody drug conjugate into cancer cells (Lewis C D, et al. Clin Cancer Res. 2021:27(11):3224-33; Adams S R, et al. Nature communications. 2016:7:13019; Hingorani D V, et al. Molecular cancer therapeutics. 2020:19(1): 157⁻⁶⁷). Alternatively, antibody-radioconjugates have great potential to achieve cytotoxicity, when alpha emitters are conjugated to the antibodies. Bispecific and fusion antibodies are gaining use in the treatment of cancer. Thus, one antibody can lead to the development of several different types of cancer drugs.

One challenge in the development of therapeutic antibodies is the paucity of cancer specific antigens. For example, cancer neoantigens contain mutations that are specific to cancer, for example mutations in EGFR. A second example is over-expressed cancer antigens such as her2/neu or CD20. Each of these examples of cancer antigens are limited to particular cancer subtypes. Moreover, only a fraction of patients have cancer that expresses these antigens: for example, only 30% of breast cancer patients show her2/neu expression. Furthermore, intra-tumoral heterogeneity is a problem in which the antigen distribution is not present on every cell within a tumor or the antigen might be inaccessible to antibody binding. Overcoming these limitations of cancer antigens can lead to improvement of the utility of therapeutic antibody treatment of cancer.

Cancer is substantially more efficient at responding to oxidative stress following ionizing radiation exposure as compared to normal tissues. The first radiation-inducible proteins to be identified include cell adhesion molecules (Hariri G, et al. Annals of Biomedical Engineering. 2008:36(5):821⁻³⁰: Shamay Y, et al. Science Translational Medicine. 2016:8(345):345ra87-ra87). These inducible proteins are not well-suited for antibody development either because they are limited to microvascular endothelial cells and not expressed on cancer cells, or they might be shed from cancer cells. In contrast, cancer cell response to radiation involving the ER stress response (ERSR) is exaggerated in cancer (Elfiky A A, Baghdady A M, Ali S A, Ahmed M I. Life Sci. 2020; 260:118317). Cancer cells are more likely to survive and propagate in the tumor microenvironment with high levels of oxidative stress due to a substantial increase in the expression of stress regulated proteins. One of the physiological responses of cancer cells to radiation is the surface expression of some of these ERSR proteins. For example, GRP78 is highly over-expressed in cancer cells and participates in cancer response to ionizing radiation (Dadey D Y A, et al. Mol Cancer Res. 2018:16(10): 1447⁻⁵³: Dadey D Y A, et al. Clin Cancer Res. 2017:23(10):2556⁻⁶⁴: Dadey D Y, et al. Oncotarget. 2016:7(2):2080⁻⁹²).

Radiation sensitizing antibodies have the potential to improve therapeutic outcomes, while attenuating treatment related adverse events like lymphocyte depletion. Therapeutic antibodies can improve cancer control while minimizing lymphopenia, which is associated with the use of concomitant myelosuppressive chemotherapy. The classic example of therapeutic antibody enhancement of radiotherapy efficacy is the use of cetuximab-which binds specifically to the EGF receptor in squamous cell carcinomas—in the treatment of head and neck cancer (Bonner J A, et al. The Lancet Oncology. 2010:11(1):21⁻⁸). Pre-clinical studies of antibody drug conjugates have also been shown to improve the cytotoxic effects of radiotherapy. Thus, therapeutic antibodies can serve as radiosensitizing drugs.

### SUMMARY

Disclosed herein are isolated binding molecules including monoclonal antibodies and human antibodies against Tax interacting protein-1 (TIP1). In certain embodiments, the antibodies are characterized as binding specifically to the PDZ domain of TIP1. Certain aspects provide for treatments of cancer including the use of antibody-drug conjugates (ADCs), RIC/ARC, toxin, and immunotherapy.

Provided for herein is an isolated antibody or antigen-binding fragment thereof comprising a binding domain that specifically binds to a Tax interacting protein-1 (TIP1). For example, a TIP1 comprising or consisting of the amino acid sequence of SEQ ID NO: 1. In certain embodiments, the binding domain comprises: a VH-CDR1 comprising an amino acid sequence identical or identical except for two or one single amino acid substitutions, deletions, or insertions to GYX₃FTSX₇W (SEQ ID NO: 30), wherein X₃ is S or R and X₇ is S or N: a VH-CDR2 comprising an amino acid sequence identical or identical except for two or one single amino acid substitutions, deletions, or insertions to IYPX₄DSDT (SEQ ID NO: 31), wherein X₄ is G or R: a VH-CDR3 comprising an amino acid sequence identical or identical except for two or one single amino acid substitution, deletion, or insertion to ARX₃X₄X₅X₆DAFDX₁₂ (SEQ ID NO: 32), wherein X₃ is Q or S, X₄ is Q or V: X₅ is G or Q, X₆ is H or absent, and X₁₂ is I or L; a VL-CDR1 comprising an amino acid sequence identical or identical except for two or one single amino acid substitutions, deletions, or insertions to ALPKRY (SEQ ID NO: 9): a VL-CDR2 comprising an amino acid sequence identical or identical except for one single amino acid substitution, deletion, or insertion to KDT (SEQ ID NO: 10); and a VL-CDR3 comprising an amino acid sequence identical or identical except for three, two, or one single amino acid substitutions, deletions, or insertions to QSTDSSASYAV (SEQ ID NO: 11).

In certain embodiments, the binding domain comprises: a VH-CDR1 comprising the amino acid sequence GYSFTSNW (SEQ ID NO: 4: [v111]): a VH-CDR2 comprising the amino acid sequence IYPGDSDT (SEQ ID NO: 5): a VH-CDR3 comprising the amino acid sequence ARQQGHDAFDI (SEQ ID NO: 6: [v111]): a VL-CDR1 comprising the amino acid sequence ALPKRY (SEQ ID NO: 9): a VL-CDR2 comprising the amino acid sequence KDT (SEQ ID NO: 10); and a VL-CDR3 comprising the amino acid sequence QSTDSSASYAV (SEQ ID NO: 11).

In certain embodiments, the binding domain comprises: a VH-CDR1 comprising the amino acid sequence GYRFTSNW (SEQ ID NO: 15: [v127]): a VH-CDR2 comprising the amino acid sequence IYPGDSDT (SEQ ID NO: 5): a VH-CDR3 comprising the amino acid sequence ARSVQDAFDL (SEQ ID NO: 17: [v127]): a VL-CDR1 comprising the amino acid sequence ALPKRY (SEQ ID NO: 9): a VL-CDR2 comprising the amino acid sequence KDT (SEQ ID NO: 10); and a VL-CDR3 comprising the amino acid sequence QSTDSSASYAV (SEQ ID NO: 11).

In certain embodiments, the binding domain comprises any of the above VH-CDR1, VH-CDR2, VH-CDR3, VL-CDR1, VL-CDR2, and VL-CDR3 amino acid sequences and comprises VH and VL amino acid sequences at least 85%, 90%, 95%, 97%, 98%, 99%, or 100% identical to reference amino acid sequences SEQ ID NO: 3 [v111] and SEQ ID NO: 8 [vill], respectively.

In certain embodiments herein, the binding domain specifically binds to the PDZ domain of TIP1. In certain embodiments, the antibody or antigen-binding fragment can inhibit binding of TIP1 to TIP1 binding proteins. In certain embodiments, binding of the binding domain to TIP1 can inhibit cancer cell migration, metastasis, and/or viability. And, in certain embodiments, binding of the binding domain to TIP1 can enhance the cytotoxicity of an anti-cancer therapy on a tumor or cancer cell.

In certain embodiments, the antibody or antigen-binding fragment thereof of this disclosure is a monoclonal antibody. In certain embodiments, the antibody or antigen-binding fragment comprises an IgG constant region or fragment thereof, for example, a human IgG1 constant region or fragment thereof.

In certain embodiments, the antibody or antigen-binding fragment thereof of this disclosure is conjugated to an anti-cancer agent, a protein, a lipid, a detectable label, and/or a polymer, or any combination thereof.

Certain embodiments provide for compositions comprising the antibody or antigen-binding fragment thereof of this disclosure and a pharmaceutically acceptable carrier and/or excipient.

Certain embodiments provide for a kit, comprising the antibody or antigen-binding fragment thereof or the composition of this disclosure and instructions for using the antibody or antigen-binding fragment thereof or using the composition or directions for obtaining instructions for using the antibody or antigen-binding fragment thereof.

Certain embodiments provide for an isolated polynucleotide comprising a nucleic acid encoding the antibody or antigen-binding fragment thereof of this disclosure or a subunit thereof. In certain embodiments, the nucleic acid encodes a VH of said antibody or antigen-binding fragment, for example wherein the VH comprises an amino acid sequence at least 85%, 90%, 95%, 97%, 98%, 99%, or 100% identical to the reference amino acid sequence SEQ ID NO: 3; and optionally, wherein the nucleic acid comprises the nucleic acid sequence of SEQ ID NO: 2. In certain embodiments, the nucleic acid encodes a VL of said antibody or antigen-binding fragment, for example wherein the nucleic acid encodes a VL, and wherein the VL comprises an amino acid sequence at least 85%, 90%, 95%, 97%, 98%, 99%, or 100% identical to the reference amino acid sequence SEQ ID NO: 8; and optionally, wherein the nucleic acid comprises the nucleic acid sequence of SEQ ID NO: 7.

Certain embodiments provide for a polynucleotide or a combination of polynucleotides encoding the antibody or antigen-binding fragment thereof of this disclosure. In certain embodiments, the polynucleotide or combination of polynucleotides comprises a nucleic acid encoding a VH and a nucleic acid encoding a VL. In certain embodiments, the nucleic acid encoding a VH and the nucleic acid encoding a VL are in the same vector. In certain embodiments, the nucleic acid encoding a VH and the nucleic acid encoding a VL are in different vectors.

Certain embodiments provide for a host cell comprising the polynucleotide or combination of polynucleotides and/or vector or vectors comprising the polynucleotide or combination of polynucleotides described herein. Certain embodiments provide for a method of making the antibody or antigen-binding fragment thereof of this disclosure, comprising culturing said host cell and isolating the antibody or antigen-binding fragment thereof.

Certain embodiments provide for a method for treating cancer in a human subject comprising administering to a human in need thereof an effective amount of the antibody or antigen-binding fragment thereof or the composition of this disclosure, e.g., use of an effective amount of the antibody or antigen-binding fragment thereof or the composition for treating cancer in a human subject. In certain embodiments, the antibody or antigen-binding fragment is co-administered and/or used with an anti-cancer therapy, and optionally, said co-administration and/or use enhances the cytotoxicity of the anti-cancer therapy on a tumor or cancer cell. In certain embodiments, the anti-cancer therapy is radiation therapy.

Certain embodiments provide for a method of detecting a tumor or cancer cell comprising administering to a subject the antibody or antigen-binding fragment thereof or the composition of this disclosure, wherein the antibody or antigen-binding fragment is conjugated to a detectable label, and optionally, performing a detection step to detect the tumor or cancer cell according to the type of detectable label conjugated to the antibody or antigen-binding fragment. e.g., use of the antibody or antigen-binding fragment thereof or the composition to detect a tumor or cancer cell, wherein the antibody or antigen-binding fragment is conjugated to a detectable label.

Provided for herein is an isolated antibody or antigen-binding fragment thereof comprising a binding domain that specifically binds to Tax interacting protein-1 (TIP1), wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 4, 5, 6, 9, 10, and 11 [v111]: SEQ ID NOs: 15, 16, 17, 12, 13, and 14 [v127]: SEQ ID NOs: 21, 22, 23, 18, 19, and 20 [v154]: or SEQ ID NOs: 27, 28, 29, 24, 25, and 26 [v144], respectively.

### BRIEF DESCRIPTION OF THE DRAWINGS

FIG. 1A-C. FIG. 1 shows: (A) DNA gel showing the amplification of full-length v111 from the phagemid: (B) Western blot showing the expression of v111 in the supernatant of Iqexpress; and (C) ELISA showing binding of v111 to TIP1. The expression supernatant was diluted three-fold and incubated with TIP1 protein for indirect ELISA assay. K_{D} was not calculated since antibody concentration is in arbitrary units.

FIG. 2A-C. FIG. 2 shows: (A) a representative TIP1 protein sequence (SEQ ID NO: 1). The boxed letters represent the predicted TIP1 interacting residues with v111 by computational docking; (B) 3D surface model of TIP1 showing the predicted interacting residues; and (C) Dot blot showing the epitope mapping of v111. Linear epitope sequence is shown in the picture.

FIG. 3. FIG. 3 is an illustration of a 3D surface model of TIP1 in green showing the predicted interacting residues as sticks. The v111 heavy chain and light chain are shown interacting with the TIP1 protein. Computational docking of TIP1 with v111 was performed using Z-dock software package.

FIG. 4. FIG. 4 is an illustration of a 3D transparent surface model of TIP1 in green showing the predicted interacting residues as sticks. The v111 heavy chain and light chain are shown interacting with the TIP1 protein. Computational docking of TIP1 with v111 was performed using Z-dock software package.

FIG. 5. FIG. 5 shows TIP1-v111 sequences:

TABLE 1

| SEQ ID NO: | Sequence for TIP1 clone v111 |
|---|---|
| 2 | Heavy Chain variable region DNA sequence |
| 3 | Heavy Chain variable region amino acid sequence |
| 4 | VH-CDR1 |
| 5 | VH-CDR2 |
| 6 | VH-CDR3 |
| 7 | Light Chain variable region DNA sequence |
| 8 | Light Chain variable region amino acid sequence |
| 9 | VL-CDR1 |
| 10 | VL-CDR2 |
| 11 | VL-CDR3 |

FIG. 6. FIG. 6 shows the CDR sequences and affinity (by Biacore) of four anti-TIP1 antibody clones:

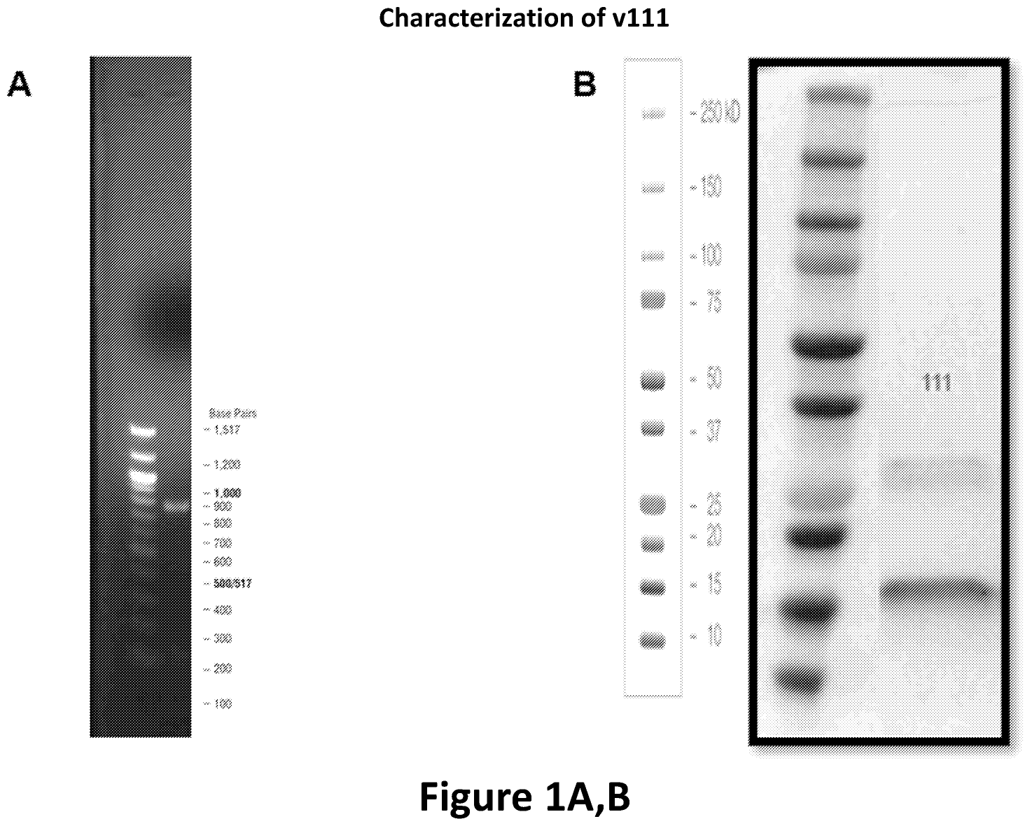

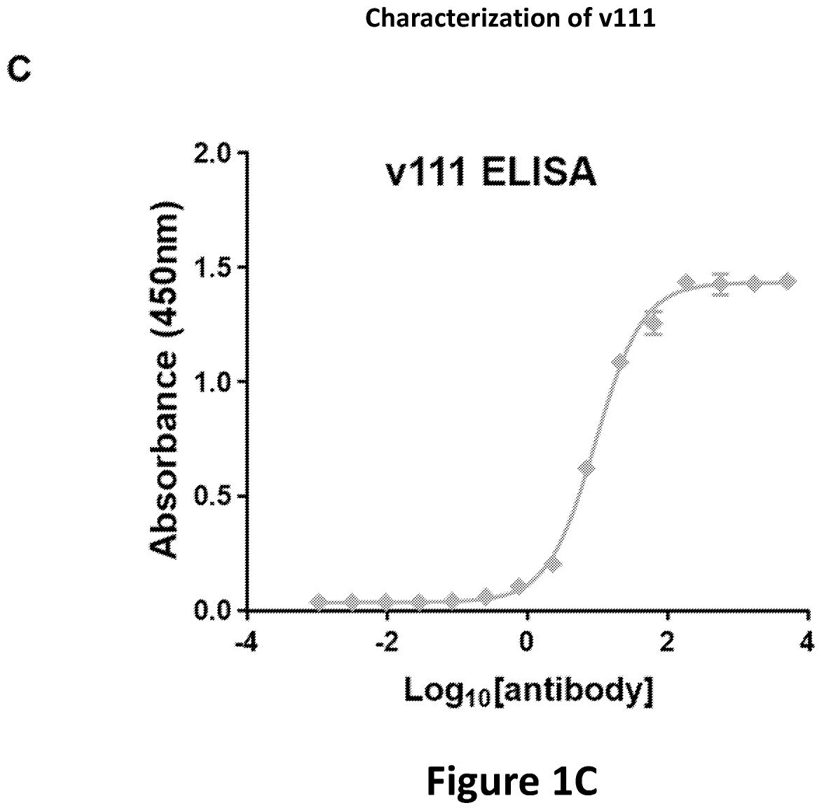

TABLE 2

| SEQ ID NO: | Sequence |
|---|---|
| 9 | TIP1 clone v111 VL-CDR1 |
| 10 | TIP1 clone v111 VL-CDR2 |
| 11 | TIP1 clone v111 VL-CDR3 |
| 4 | TIP1 clone v111 VH-CDR1 |
| 5 | TIP1 clone v111 VH-CDR2 |
| 6 | TIP1 clone v111 VH-CDR3 |
| 12 | TIP1 clone v127 VL-CDR1 |
| 13 | TIP1 clone v127 VL-CDR2 |
| 14 | TIP1 clone v127 VL-CDR3 |
| 15 | TIP1 clone v127 VH-CDR1 |
| 16 | TIP1 clone v127 VH-CDR2 |
| 17 | TIP1 clone v127 VH-CDR3 |
| 18 | TIP1 clone v154 VL-CDR1 |
| 19 | TIP1 clone v154 VL-CDR2 |
| 20 | TIP1 clone v154 VL-CDR3 |
| 21 | TIP1 clone v154 VH-CDR1 |
| 22 | TIP1 clone v154 VH-CDR2 |
| 23 | TIP1 clone v154 VH-CDR3 |
| 24 | TIP1 clone v144 VL-CDR1 |
| 25 | TIP1 clone v144 VL-CDR2 |
| 26 | TIP1 clone v144 VL-CDR3 |
| 27 | TIP1 clone v144 VH-CDR1 |
| 28 | TIP1 clone v144 VH-CDR2 |
| 29 | TIP1 clone v144 VH-CDR3 |

FIG. 7. FIG. 7 shows Purified human anti-TIP1 full length IgGs were resolved on SDS PAGE under non-reducing (left) and reducing condition (right). Intact full length IgGs are observed above 150 kDa in the non-reducing conditions. In the reducing condition, heavy chains and light chains are observed at 50 kDa and 25 kDa respectively.

FIG. 8A-C. FIG. 8A-C shows the results of antibody purification using protein A and size exclusion chromatography.

FIG. 9A,B. FIG. 9 shows results of recombinant TIP1 protein coated on ELISA plates. Three-fold serial dilutions (starting at 20 nM) of the purified human anti-TIP1 full length IgG1 antibodies were incubated with the protein. Anti-human HRP conjugated antibody was used as the detection antibody along with TMB substrate. Absorbance at 450 nm vs concentration is plotted in the graph. The data was fitted using the “One-site specific” (A) and log(agonist) vs. response (B)—Variable slope (four parameters) model in GraphPad Prism software. The K_{D} and EC50 values are shown in the table below each graph.

FIG. 10. FIG. 10 shows results of recombinant TIP1 protein (ligand) immobilized on the surface of CM5 sensor chip. Reference surface was prepared and blocked. Various concentrations of antibodies (as indicated) were passed over the ligand. Reference subtracted sensograms were fitted using the BIAevaluation software and on-rates, off-rates and KD calculated.

FIG. 11. FIG. 11 shows the molecular weight, pI and ext. coefficient of the purified v111 antibody.

FIG. 12. FIG. 12 shows whole cell lysates or purified TIP1 protein resolved on SDS-PAGE and blotted n PVDF membranes. After blocking, membranes were probed with the indicated anti-TIP1 human antibodies and developed with anti-human Fc specific HRP conjugated antibody.

FIG. 13. FIG. 13 shows the flow cytometry results and gating parameters of irradiated H460 human lung cancer cells following incubation with anti-TIP1 antibody v111. v111 binds to irradiated H460 cells at micromolar concentrations.

FIG. 14. FIG. 14 shows the flow cytometry results and gating parameters of irradiated A549 human lung cancer cells following incubation with anti-TIP1 antibody v111. v111 binds to irradiated A549 cells at micromolar concentrations.

FIG. 15A,B. FIG. 15 shows the concentration curve of v111 antibody binding to H460 (A) and A549 (B) cells. v111 binds to irradiated cancer cells at micromolar concentrations.

FIG. 16A,B. FIG. 16 shows (A) A549 cells were seeded and either sham irradiated of irradiated with three doses of 3 Gy. v111 labeled with pHrodo dye was added and imaged with a florescent microscope. Nuclei were stained with Nucblue stain. Red punctate staining indicated accumulation of the antibody in intracellular acidic compartments. (B) Bar graph showing the staining intensity in the red channel from three independent images.

FIG. 17. FIG. 17 shows v111 that was incubated in plasma samples from three healthy volunteers at 37° C. and collected at the indicated time points. ELISA assay was used to evaluate stability of the antibody over ten days.

FIG. 18. FIG. 18 shows v111 that was injected into three nude mice and plasma collected at the indicated time points by sub-mandibular bleeding. ELISA assay was used to evaluate stability of the antibody over ten days.

FIG. 19A-C. FIG. 19 shows colony formation assay with human TIP1 antibodies v111 in A549 cells. (A) shows the treatment schema. (B) shows the surviving fraction with two doses of v111 (50 and 100 ug/ml). (C) shows representative pictures of colonies observed after treatment. Abortive colonies (black arrows) were observed with antibody treatment.

FIG. 20A,B. FIG. 20 shows Whole body NIR imaging of v111 anti-TIP1 human antibody in nude mice bearing H460 tumors: (A) Representative NIR images of nude mice at the indicated time points after tail-vein injection of the IRDye 800 labeled v111 antibody. Mice have H460 tumors in the right hind limb: (B) Normalized NIR signal intensities of the tumors over ten days.

FIG. 21A-D. FIG. 21 shows whole body NIR imaging of v111 anti-TIP1 human antibody in nude mice bearing A549 tumors: (A) Representative NIR images of nude mice at the indicated time points after tail-vein injection of the IRDye 800 labeled v111 antibody. Mice have A549 tumors in both hind limbs. The tumors on the right hind limb were irradiated with three doses of 3 Gy radiation and the tumors on the left hindlimb served as un-irradiated (sham) controls: (B) Normalized NIR signal intensities of the IR vs sham tumors: (C) Mice were euthanized after 7 days of antibody injection and vital organs harvested for biodistribution. Shown are the images of the harvested organs from one representative mouse: (D) NIR signal intensities of harvested organs.

### DETAILED DESCRIPTION

### Definitions

The term “a” or “an” entity refers to one or more of that entity: for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

Unless defined otherwise, technical and scientific terms used herein have the same meaning as commonly understood by one of ordinary skill in the art to which this disclosure is related.

Where applicable, units, prefixes, and symbols are denoted in their Système International de Unites (SI) accepted form.

Numeric ranges are inclusive of the numbers defining the range.

Unless otherwise indicated, amino acid sequences are written left to right in amino (N) to carboxy (C) orientation and nucleic acid sequences are written from 5′ to 3′, left to right.

The headings provided herein are not limitations of the various aspects and embodiments of the disclosure, which can be had by reference to the specification as a whole.

Terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of amino acid monomers linearly linked by peptide bonds (also known as amide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, hydrophobic interactions, etc., to produce, e.g., a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring.” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, insertions, and/or deletions. Derivatives are polypeptides that have been altered so as to exhibit additional features not found on the native polypeptide, such as increased resistance to proteolytic degradation. Examples include fusion proteins. Variant polypeptides can also be referred to herein as “polypeptide analogs.” As used herein a “derivative” also refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline: 5-hydroxylysine can be substituted for lysine: 3-methylhistidine can be substituted for histidine: homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993): Kobayashi et al., Protein Eng. 12(10):879⁻⁸⁸⁴ (1999); and Burks et al., Proc. Natl. Acad. Sci. USA 94: 412⁻⁴¹⁷ (1997)).

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

Disclosed herein are certain binding molecules, or antigen-binding fragments, variants and/or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

The terms “antibody” and “immunoglobulin” can be used interchangeably herein. An antibody (including a fragment, variant, or derivative thereof) as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Antibodies of this disclosure include scFv-Fc fusion antibodies and bispecific antibodies.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region: the CH3 and CL domains actually comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen-binding site present at the end of each arm of the Y. More specifically, the antigen-binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the γ-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901⁻⁹¹⁷ (1987), which are incorporated herein by reference in their entireties). Immunoglobulin variable domains can also be analyzed using the IMGT information system (www://imgt.cines.fr/) (IMGTR/V-Quest) to identify variable region segments, including CDRs. See, e.g., Brochet, X. et al., Nucl. Acids Res. 36:W503⁻⁵⁰⁸ (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

Antibodies and antigen-binding fragments thereof, variants and/or derivatives thereof, include but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or antigen-binding fragment thereof binds to an epitope via its antigen binding domain, and that the binding entails some recognition between the antigen binding domain and the epitope. According to this definition, a binding molecule is said to “specifically bind” to an epitope when it binds to that epitope, via its antigen-binding domain binds more readily than it would bind to a random, unrelated epitope.

An antibody or antigen-binding fragment thereof, including a variant or derivative thereof, can be said to competitively inhibit binding of a reference antibody or antigen-binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. As used herein, the term “avidity” refers to the overall stability of the complex between a population of immunoglobulins and an antigen, that is, the functional combining strength of an immunoglobulin mixture with the antigen. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen: a measure of relatedness between two different antigenic substances. Thus, a binding molecule is cross-reactive if it binds to an epitope other than the one that induced its formation, e.g., the PDZ domain of TIP1. The cross-reactive epitope contains many of the same complementary structural features as the inducing epitope, and in some cases, can actually fit better than the original.

A binding molecule, e.g., an antibody or antigen-binding fragment, variant, or derivative thereof can also be described or specified in terms of their binding affinity to an antigen. For example, a binding molecule can bind to an antigen with a dissociation constant or K_{D} no greater than 5×10⁻⁵ M, 1×10⁻⁵ M, 5×10⁻⁶ M, 1×10⁻⁶ M, 5×10⁻⁷ M, 1×10⁻⁷ M, 5×10⁻⁸ M, Is 10⁻⁸ M, 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵ M, or 1×10⁻¹⁵ M.

“Immunogenicity” as used herein refers to an unwanted immune response by the recipient against a therapeutic antibody. This reaction leads to production of anti-drug-antibodies (ADAs), inactivating the therapeutic effects of the treatment and potentially inducing adverse effects. T cell epitope content is one of the factors that contributes to antigenicity. Likewise, T Cell epitopes can cause unwanted immunogenicity, including the development of ADAs. A key determinant in T cell epitope immunogenicity is the binding strength of T cell epitopes to major histocompatibility complexes (MHC or HLA) molecules. Epitopes with higher binding affinities are more likely to be displayed on the surface of a cell. Because a T cell's T cell receptor recognizes a specific epitope, only certain T cells are able to respond to a certain peptide bound to MHC on a cell surface. When monoclonal antibodies are administered, antigen presenting cells (APCs), such as a B cell or Dendritic Cell, will present these substances as peptides, which T cells may recognize. This may result in unwanted immunogenicity. T cell epitope content is measured using in silico tools. Immunoinformatics algorithms for identifying T-cell epitopes are triaged into higher risk and lower risk categories. These categories refer to assessing and analyzing whether an immunotherapy or vaccine will cause unwanted immunogenicity. One approach is to parse protein sequences into overlapping nonamer (that is, 9 amino acid) peptide frames, each of which is then evaluated for binding potential to each of six common class I HLA alleles that “cover” the genetic backgrounds of most humans worldwide. By calculating the density of high-scoring frames within a protein, a protein's overall “immunogenicity score” can be estimated.

Antibody fragments including single-chain antibodies can comprise the variable region(s) alone or in combination with the entirety or a portion of the following: hinge region, CH1, CH2, and CH3 domains. Also included are antigen-binding fragments that comprise any combination of variable region(s) with a hinge region, CH1, CH2, and CH3 domains. Binding molecules, e.g., antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described for example in, U.S. Pat. No. 5,939,598 by Kucherlapati et al., which is incorporate herein by reference.

As used herein, the term “heavy chain portion” includes amino acid sequences derived from an immunoglobulin heavy chain, a binding molecule, e.g., an antibody comprising a heavy chain portion comprises at least one of: a CH1 domain, a hinge (e.g., upper, middle, and/or lower hinge region) domain, a CH2 domain, a CH3 domain, or a variant or fragment thereof. For example, a binding molecule, e.g., an antibody or fragment, variant, or derivative thereof can comprise a polypeptide chain comprising a CH1 domain: a polypeptide chain comprising a CH1 domain, at least a portion of a hinge domain, and a CH2 domain: a polypeptide chain comprising a CH1 domain and a CH3 domain: a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a binding molecule, e.g., an antibody or fragment, variant, or derivative thereof comprises a polypeptide chain comprising a CH3 domain. Further, a binding molecule for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a binding molecule, e.g., an antibody as disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion comprises at least one of a VL or CL domain.

Antibodies or antigen-binding fragments, including variants and/or derivatives thereof disclosed herein can be described or specified in terms of the epitope(s) or portion(s) of an antigen, that they recognize or specifically bind. The portion of a target antigen that specifically interacts with the antigen-binding domain of an antibody is an “epitope,” or an “antigenic determinant.” A target antigen, e.g., a PDZ domain of TIP1 comprises at least a single epitope, but typically comprises at least two epitopes, and can include any number of epitopes, depending on the size, conformation, and type of antigen.

As used herein, the term “chimeric antibody” means any antibody wherein the immunoreactive region or site is obtained or derived from a first species and the constant region (which can be intact, partial, or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single binding molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387⁻⁹⁴ (2010): Mabry and Snavely, IDrugs. 13:543-9 (2010)).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring.” or that are, or that might be at any time, determined or interpreted by a judge or an administrative or judicial body to be, “naturally-occurring.”

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide as disclosed herein, e.g., a polynucleotide encoding a polypeptide subunit provided herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

A “vector” is nucleic acid molecule used as a vehicle to manipulate and utilize genetic material. For example, a vector can be introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” “treatment of,” and the like (e.g., in the phrase “treating a subject”) refers to reducing the potential for disease pathology and/or reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

The term “pharmaceutical composition” refers to a preparation that is in such form as to permit the biological activity of the active ingredient to be effective and does not contain components that are unacceptably toxic to a subject to which the composition would be administered. Such composition can be sterile.

An “effective amount” as disclosed herein is an amount sufficient to carry out a specifically stated purpose. An “effective amount” can be determined empirically and in a routine manner, in relation to the stated purpose.

A consensus sequence of a CDR region can be determined for purposes of this disclosure by aligning CDR sequences from multiple antibodies, for example, the VH-CDR1 amino acid sequences of the v111, v127, and v154 clones:

v111
(SEQ ID NO: 4)
GYSFTSNW
v127
(SEQ ID NO: 15)
GYRFTSNW
v154
(SEQ ID NO: 21)
GYSFTSSW
Consensus
(SEQ ID NO: 30)
GYX₃FTSX₇W

wherein X₃ is S or R and X₇ is S or N.

The consensus sequences of this disclosure include:

VH-CDR1
(SEQ ID NO: 30)
GYX₃FTSX₇W,
wherein X₃ is S or R and X₇ is S or N;
VH-CDR2
(SEQ ID NO: 31)
IYPX₄DSDT,
wherein X₄ is G or R;
and
VH-CDR3
(SEQ ID NO: 32)
ARX₃X₄X₅X₆DAFDX_{12,}
wherein X₃ is Q or S, X₄ is Q or V; X₅ is G or Q,
X₆ is H or absent, and X₁₂ is I or L.

### Overview

Similar to cetuximab, antibodies to radiation-inducible proteins (e.g., GRP78 and TIP1), can enhance the cytotoxic effects of radiotherapy and improve survival in mouse models of cancer (Dadey D Y A, et al. Clin Cancer Res. 2017:23(10):2556-64). Cancer cell surface expression of these stress-regulated proteins increases their accessibility to antibody binding during radiotherapy. TIP1 is a radiation inducible antigen discovered by phage display peptide library. TIP-1 expression levels correlate with poor prognosis in human cancers (Wang H, et al. Oncogene. 2014:33(12): 1558-69). During the stress response to radiation, TIP1 is translocated to the cancer cell surface where it is accessible to antibody binding (Yan H, et al. Oncotarget. 2016:7(28): 43352-62).

Tax interacting protein-1 (TIP1) is a scaffold protein that attaches to the cell membrane and anchors proteins involved in signal transduction. It is highly overexpressed in cancer and expression levels correlate strongly with worsened outcome in cancer patients. It has been previously shown that it is involved in cancer cell migration, metastasis, and viability (Wang H, et al. Oncogene. 2014:33(12): 1558-69) Presently, it has been discovered that the protein binding domain of TIP1 is its PDZ domain, which appears essential for its biological function. It is also been reported that antibodies that dissociate TIP1's interaction with its binding proteins induce cytotoxicity and enhance the efficacy of cancer therapy.

Disclosed herein is the discovery of antibodies to this inducible antigen to enhance the efficacy of radiotherapy. Antibodies to the functional domain (PDZ domain) of TIP1 disrupted its interaction with binding partners, leading to reduced cancer cell viability and radiosensitization. Tax-interacting protein-1 (TIP1) Antibodies

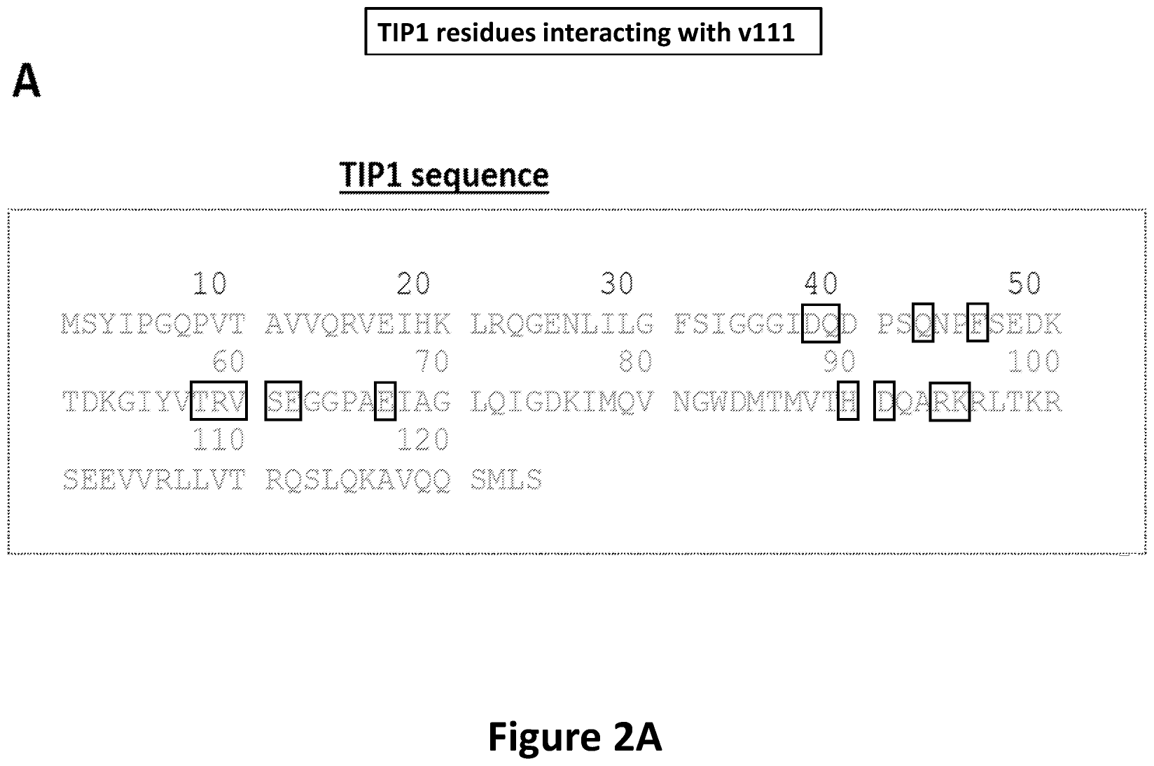

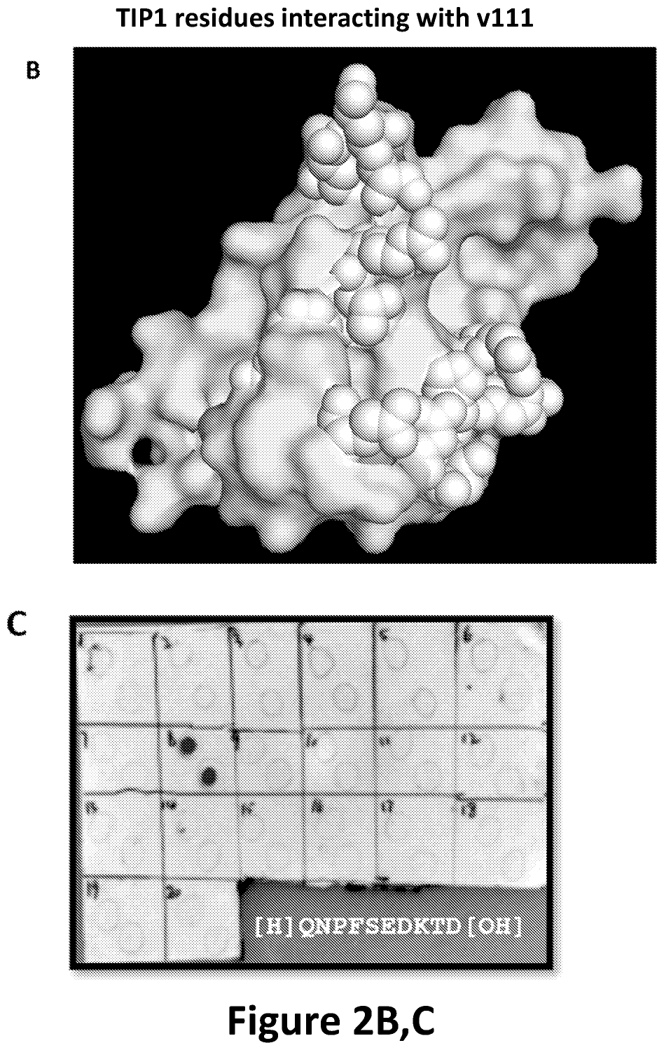

This disclosure provides for an isolated antibody or antigen-binding fragment thereof comprising a binding domain that specifically binds to Tax interacting protein-1 (TIP1) (e.g., SEQ ID NO: 1: FIG. 2A). One of ordinary skill in the art will know that antibody binding domains can be characterized by six complementarity-determining regions (CDRs), with three occurring within the heavy chain variable region (VH), commonly referred to as VH-CDR1, VH-CDR2, and VH-CDR3, and with three occurring within the light chain variable region (VL), commonly referred to as VL-CDR1, VL-CDR2, and VL-CDR 3. In certain embodiments, the binding domain of this disclosure comprises:
(i) a VH-CDR1 comprising an amino acid sequence identical or identical except for two or one single amino acid substitutions, deletions, or insertions to GYX₃FTSX₇W (SEQ ID NO: 30), wherein X₃ is S or R and X₇ is S or N,
optionally, a VH-CDR1 comprising an amino acid sequence identical or identical except for one single amino acid substitution, deletion, or insertion to GYX₃FTSX₇W (SEQ ID NO: 30), wherein X₃ is S or R and X₇ is S or N,
further optionally, a VH-CDR1 comprising an amino acid sequence identical to GYX₃FTSX₇W (SEQ ID NO: 30), wherein X₃ is S or R and X₇ is S or N;
(ii) a VH-CDR2 comprising an amino acid sequence identical or identical except for two or one single amino acid substitutions, deletions, or insertions to IYPX₄DSDT (SEQ ID NO: 31), wherein X₄ is G or R,
optionally, a VH-CDR2 comprising an amino acid sequence identical or identical except for one single amino acid substitution, deletion, or insertion to IYPX₄DSDT (SEQ ID NO: 31), wherein X₄ is G or R,
further optionally, a VH-CDR2 comprising an amino acid sequence identical to IYPX₄DSDT (SEQ ID NO: 31), wherein X₄ is G or R;
(iii) a VH-CDR3 comprising an amino acid sequence identical or identical except for one single amino acid substitution, deletion, or insertion to ARX₃X₄X₅X₆DAFDX₁₂ (SEQ ID NO: 32), wherein X₃ is Q or S, X₄ is Q or V: X₅ is G or Q, X₆ is H or absent, and X₁₂ is I or L,
optionally, a VH-CDR3 comprising an amino acid sequence identical to ARX₃X₄X₅X₆DAFDX₁₂ (SEQ ID NO: 32), wherein X₃ is Q or S, X₄ is Q or V: X₅ is G or Q, X₆ is H or absent, and X₁₂ is I or L;
(iv) a VL-CDR1 comprising an amino acid sequence identical or identical except for two or one single amino acid substitutions, deletions, or insertions to ALPKRY (SEQ ID NO: 9),
optionally, a VL-CDR1 comprising an amino acid sequence identical or identical except for one single amino acid substitution, deletion, or insertion to ALPKRY (SEQ ID NO: 9),
further optionally, a VL-CDR1 comprising an amino acid sequence identical to ALPKRY (SEQ ID NO: 9);
(v) a VL-CDR2 comprising an amino acid sequence identical or identical except for one single amino acid substitutions, deletions, or insertions to KDT (SEQ ID NO: 10),
optionally, a VL-CDR2 comprising an amino acid sequence identical to KDT (SEQ ID NO: 10); and
(vi) a VL-CDR3 comprising an amino acid sequence identical or identical except for three, two, or one single amino acid substitutions, deletions, or insertions to QSTDSSASYAV (SEQ ID NO: 11),
optionally, a VL-CDR3 comprising an amino acid sequence identical or identical except for two, or one single amino acid substitutions, deletions, or insertions to QSTDSSASYAV (SEQ ID NO: 11),
further optionally, a VL-CDR3 comprising an amino acid sequence identical or identical except for one single amino acid substitution, deletion, or insertion to QSTDSSASYAV (SEQ ID NO: 11),
further optionally, a VL-CDR3 comprising an amino acid sequence identical to QSTDSSASYAV (SEQ ID NO: 11).

In certain embodiments of any or all of the above CDRs, the amino acid substitutions, deletions, or insertions are limited to just amino acid substitutions, just amino acid deletions, or just amino acid insertions.

In certain embodiments, the binding domain comprises: a VL-CDR1 comprising the amino acid sequence ALPKRY (SEQ ID NO: 9): a VL-CDR2 comprising the amino acid sequence KDT (SEQ ID NO: 10); and/or a VL-CDR3 comprising the amino acid sequence QSTDSSASYAV (SEQ ID NO: 11). In certain embodiments, the binding domain comprises: a VL-CDR1 comprising the amino acid sequence ALPKRY (SEQ ID NO: 9): a VL-CDR2 comprising the amino acid sequence KDT (SEQ ID NO: 10); and a VL-CDR3 comprising the amino acid sequence QSTDSSASYAV (SEQ ID NO: 11).

In certain embodiments, the binding domain comprises: a VH-CDR1 comprising the amino acid sequence GYX₃FTSX₇W (SEQ ID NO: 30), wherein X₃ is S or R and X₇ is S or N: a VH-CDR2 comprising the amino acid sequence IYPX₄DSDT (SEQ ID NO: 31), wherein X₄ is G or R; and/or a VH-CDR3 comprising the amino acid sequence ARX₃X₄X₅X₆DAFDX₁₂ (SEQ ID NO: 32), wherein X₃ is Q or S. X₄ is Q or V: X₅ is G or Q. X₆ is H or absent, and X₁₂ is I or L. In certain embodiments, the binding domain comprises: a VH-CDR1 comprising the amino acid sequence GYX₃FTSX₇W (SEQ ID NO: 30), wherein X₃ is S or R and X₇ is S or N: a VH-CDR2 comprising the amino acid sequence IYPX₄DSDT (SEQ ID NO: 31), wherein X₄ is G or R; and a VH-CDR3 comprising the amino acid sequence ARX₃X₄X₅X₆DAFDX₁₂ (SEQ ID NO: 32), wherein X₃ is Q or S. X₄ is Q or V; X₅ is G or Q, X₆ is H or absent, and X₁₂ is I or L.

In certain embodiments, the binding domain comprises: a VH-CDR1 comprising the amino acid sequence GYSFTSNW (SEQ ID NO: 4: [v111]) or GYRFTSNW (SEQ ID NO: 15; [v127]): a VH-CDR2 comprising the amino acid sequence IYPGDSDT (SEQ ID NO: 5); and/or a VH-CDR3 comprising the amino acid sequence ARX₃X₄X₅X₆DAFDX₁₂ (SEQ ID NO: 32), wherein X₃ is Q or S. X₄ is Q or V: X₅ is G or Q. X₆ is H or absent, and X₁₂ is I or L. In certain embodiments, the binding domain comprises: a VH-CDR1 comprising the amino acid sequence GYSFTSNW (SEQ ID NO: 4: [v111]) or GYRFTSNW (SEQ ID NO: 15: [v127]); a VH-CDR2 comprising the amino acid sequence IYPGDSDT (SEQ ID NO: 5); and a VH-CDR3 comprising the amino acid sequence ARX₃X₄X₅X₆DAFDX₁₂ (SEQ ID NO: 32), wherein X₃ is Q or S. X₄ is Q or V: X₅ is G or Q. X₆ is H or absent, and X₁₂ is I or L.

In certain embodiments, the binding domain comprises: a VH-CDR1 comprising the amino acid sequence GYSFTSNW (SEQ ID NO: 4: [v111]) or GYRFTSNW (SEQ ID NO: 15; [v127]): a VH-CDR2 comprising the amino acid sequence IYPGDSDT (SEQ ID NO: 5); and/or a VH-CDR3 comprising the amino acid sequence ARQQGHDAFDI (SEQ ID NO: 6: [v111]) or ARSVQDAFDL (SEQ ID NO: 17: [v127]). In certain embodiments, the binding domain comprises: a VH-CDR1 comprising the amino acid sequence GYSFTSNW (SEQ ID NO: 4; [v111]) or GYRFTSNW (SEQ ID NO: 15: [v127]): a VH-CDR2 comprising the amino acid sequence IYPGDSDT (SEQ ID NO: 5); and a VH-CDR3 comprising the amino acid sequence ARQQGHDAFDI (SEQ ID NO: 6: [v111]) or ARSVQDAFDL (SEQ ID NO: 17: [v127]). In certain embodiments, the binding domain comprises: a VH-CDR1 comprising the amino acid sequence GYSFTSNW (SEQ ID NO: 4: [v111]): a VH-CDR2 comprising the amino acid sequence IYPGDSDT (SEQ ID NO: 5); and/or a VH-CDR3 comprising the amino acid sequence ARQQGHDAFDI (SEQ ID NO: 6: [v111]). In certain embodiments, the binding domain comprises: a VH-CDR1 comprising the amino acid sequence GYSFTSNW (SEQ ID NO: 4; [v111]): a VH-CDR2 comprising the amino acid sequence IYPGDSDT (SEQ ID NO: 5); and a VH-CDR3 comprising the amino acid sequence ARQQGHDAFDI (SEQ ID NO: 6: [v111]). In certain embodiments, the binding domain comprises: a VH-CDR1 comprising the amino acid sequence GYRFTSNW (SEQ ID NO: 15: [v127]): a VH-CDR2 comprising the amino acid sequence IYPGDSDT (SEQ ID NO: 5); and/or a VH-CDR3 comprising the amino acid sequence ARSVQDAFDL (SEQ ID NO: 17: [v127]). In certain embodiments, the binding domain comprises: a VH-CDR1 comprising the amino acid sequence GYRFTSNW (SEQ ID NO: 15; [v127]): a VH-CDR2 comprising the amino acid sequence IYPGDSDT (SEQ ID NO: 5); and a VH-CDR3 comprising the amino acid sequence or ARSVQDAFDL (SEQ ID NO: 17: [v127]). In certain embodiments, the binding domain comprises: a VH-CDR1 comprising the amino acid sequence GYSFTSNW (SEQ ID NO: 4: [v111]); a VH-CDR2 comprising the amino acid sequence IYPGDSDT (SEQ ID NO: 5); and/or a VH-CDR3 comprising the amino acid sequence ARSVQDAFDL (SEQ ID NO: 17: [v127]). In certain embodiments, the binding domain comprises: a VH-CDR1 comprising the amino acid sequence GYSFTSNW (SEQ ID NO: 4; [v111]); a VH-CDR2 comprising the amino acid sequence IYPGDSDT (SEQ ID NO: 5); and a VH-CDR3 comprising the amino acid sequence ARSVQDAFDL (SEQ ID NO: 17: [v127]). In certain embodiments, the binding domain comprises: a VH-CDR1 comprising the amino acid sequence GYRFTSNW (SEQ ID NO: 15: [v127]): a VH-CDR2 comprising the amino acid sequence IYPGDSDT (SEQ ID NO: 5); and/or a VH-CDR3 comprising the amino acid sequence ARQQGHDAFDI (SEQ ID NO: 6: [v111]). In certain embodiments, the binding domain comprises: a VH-CDR1 comprising the amino acid sequence GYRFTSNW (SEQ ID NO: 15; [v127]): a VH-CDR2 comprising the amino acid sequence IYPGDSDT (SEQ ID NO: 5); and a VH-CDR3 comprising the amino acid sequence ARQQGHDAFDI (SEQ ID NO: 6: [v111]).

In certain embodiments, the binding domain comprises: a VH-CDR1 comprising the amino acid sequence GYSFTSNW (SEQ ID NO: 4: [v111]): a VH-CDR2 comprising the amino acid sequence IYPGDSDT (SEQ ID NO: 5): a VH-CDR3 comprising the amino acid sequence ARQQGHDAFDI (SEQ ID NO: 6: [v111]); a VL-CDR1 comprising the amino acid sequence ALPKRY (SEQ ID NO: 9); a VL-CDR2 comprising the amino acid sequence KDT (SEQ ID NO: 10); and/or a VL-CDR3 comprising the amino acid sequence QSTDSSASYAV (SEQ ID NO: 11). In certain embodiments, the binding domain comprises all six CDRs of the v111 clone disclosed herein, i.e.: a VH-CDR1 comprising the amino acid sequence GYSFTSNW (SEQ ID NO: 4: [v111]): a VH-CDR2 comprising the amino acid sequence IYPGDSDT (SEQ ID NO: 5); a VH-CDR3 comprising the amino acid sequence ARQQGHDAFDI (SEQ ID NO: 6: [v111]): a VL-CDR1 comprising the amino acid sequence ALPKRY (SEQ ID NO: 9): a VL-CDR2 comprising the amino acid sequence KDT (SEQ ID NO: 10); and a VL-CDR3 comprising the amino acid sequence QSTDSSASYAV (SEQ ID NO: 11).

In certain embodiments, the binding domain comprises: a VH-CDR1 comprising the amino acid sequence GYRFTSNW (SEQ ID NO: 15: [v127]): a VH-CDR2 comprising the amino acid sequence IYPGDSDT (SEQ ID NO: 5): a VH-CDR3 comprising the amino acid sequence ARSVQDAFDL (SEQ ID NO: 17: [v127]): a VL-CDR1 comprising the amino acid sequence ALPKRY (SEQ ID NO: 9): a VL-CDR2 comprising the amino acid sequence KDT (SEQ ID NO: 10); and/or a VL-CDR3 comprising the amino acid sequence QSTDSSASYAV (SEQ ID NO: 11). In certain embodiments, the binding domain comprises all six of the CDRs of the v127 clone disclosed herein. i.e.: a VH-CDR1 comprising the amino acid sequence GYRFTSNW (SEQ ID NO: 15: [v127]): a VH-CDR2 comprising the amino acid sequence IYPGDSDT (SEQ ID NO: 5): a VH-CDR3 comprising the amino acid sequence ARSVQDAFDL (SEQ ID NO: 17: [v127]): a VL-CDR1 comprising the amino acid sequence ALPKRY (SEQ ID NO: 9): a VL-CDR2 comprising the amino acid sequence KDT (SEQ ID NO: 10); and a VL-CDR3 comprising the amino acid sequence QSTDSSASYAV (SEQ ID NO: 11).

In certain embodiments, a binding domain of an antibody or antigen-binding fragment of this disclosure comprising the VH-CDR1. VH-CDR2. VH-CDR3. VL-CDR1. VL-CDR2, and VL-CDR3 amino acid sequences disclosed herein comprises VH and VL amino acid sequences at least 85%. 90%, 95%. 97%, 98%, 99%, or 100% identical to reference amino acid sequences SEQ ID NO: 3 [v111] and SEQ ID NO: 8 [v111], respectively. In certain embodiments, a binding domain of an antibody or antigen-binding fragment of this disclosure comprising the VH-CDR1. VH-CDR2. VH-CDR3. VL-CDR1. VL-CDR2, and VL-CDR3 amino acid sequences disclosed herein comprises a VH amino acid sequence at least 85%. 90%, 95%. 97%, 98%, 99%, or 100% identical to reference amino acid sequence SEQ ID NO: 3 [v111] and a VL amino acid sequence 100% identical to SEQ ID NO: 8 [v111]. In certain embodiments, a binding domain of an antibody or antigen-binding fragment of this disclosure comprising the VH-CDR1. VH-CDR2. VH-CDR3. VL-CDR1. VL-CDR2, and VL-CDR3 amino acid sequences disclosed herein comprises a VH amino acid sequence 100% identical to SEQ ID NO: 3 [v111] and a VL amino acid sequence at least 85%, 90%, 95%, 97%, 98%, 99%, or 100% identical to reference amino acid sequence SEQ ID NO: 8 [v111]. In certain embodiments, a binding domain of an antibody or antigen-binding fragment of this disclosure comprising the VH-CDR1, VH-CDR2, VH-CDR3, VL-CDR1, VL-CDR2, and VL-CDR3 amino acid sequences disclosed herein comprises a VH amino acid sequence 100% identical to SEQ ID NO: 3 [v111] and a VL amino acid sequence 100% identical to SEQ ID NO: 8 [v111].

In certain embodiments of an antibody or antigen-binding fragment thereof, the antibody or antigen-binding fragment specifically binds to TIP1 with a K_{D} value no greater than 9×10⁻⁷, no greater than 8.8×10⁻⁷, no greater than 5×10⁻⁷, no greater than 1×10⁻⁷, no greater than 5×10⁻⁸, no greater than 1×10⁻⁸, no greater than 5×10⁻⁹, no greater than 1×10⁻⁹, no greater than 5×10⁻¹⁰, no greater than 1×10⁻¹⁰, no greater than 5×10⁻¹¹, no greater than 1×10⁻¹¹, no greater than 5×10⁻¹², and/or no greater than 1×10⁻¹². In certain embodiments of an antibody or antigen-binding fragment thereof, the antibody or antigen-binding fragment specifically binds to TIP1 with a K_{D} value of between any of 9×10⁻⁷, 8.8×10⁻⁷, 5×10⁻⁷, 1×10⁻⁷, 5×10⁻⁸, 1×10⁻⁸, 5×10⁻⁹, 1×10⁻⁹, 5×10⁻¹⁰, 1×10⁻¹⁰, 5×10⁻¹¹, 1×10⁻¹¹, or 5×10⁻¹² and any of 9×10⁻⁷, 8.8×10⁻⁷, 5×10⁻⁷, 1×10⁻⁷, 5×10⁻⁸, 1×10⁻⁸, 5×10⁻⁹, 1×10⁻⁹, 5×10⁻¹⁰, 1×10⁻¹⁰, 5×10⁻¹¹, 1×10⁻¹¹, 5×10⁻¹², or 1×10⁻¹².

In certain embodiments of an antibody or antigen-binding fragment thereof the antibody or antigen-binding fragment has an immunogenicity score of less than 1000, less than 900, less than 800, less than 700, less than 600, less than 500, less 400, and/or less 300.

As described in more detail elsewhere herein, it has been discovered that the protein binding domain of TIP1 is its PDZ domain, which appears essential for its biological function. In certain embodiments, the binding domain of the antibody or antigen-binding fragment thereof of this disclosure specifically binds to the PDZ domain of TIP1. In certain embodiments, the antibody or antigen-binding fragment can inhibit binding of TIP1 to TIP1 binding proteins. For example, without being bound by theory, it is thought that such antibodies can displace TIP1 from the metalloprotease Adam 10 which cleaves Ephrin ligand to activate endocytosis. In certain embodiments, binding of the binding domain to TIP1 inhibits cancer cell migration, metastasis, and/or viability. And, in certain embodiments, binding of the binding domain to TIP1 enhances the cytotoxicity of an anti-cancer therapy on a tumor or cancer cell.

In certain embodiments, the antibody or antigen-binding fragment thereof of as provided herein can be, for example, a humanized antibody, a chimeric antibody, or an antigen-binding fragment thereof. In certain embodiments, the antibody or antigen-binding fragment thereof is a humanized antibody or an antigen-binding fragment thereof. Moreover, the antibody or antigen-binding fragment thereof can be a monoclonal antibody, a component of a polyclonal antibody mixture, a recombinant antibody, a multi-specific antibody, or any combination thereof. In certain embodiments, the antibody or antigen-binding fragment thereof is a monoclonal antibody.

In certain embodiments, the antibody or antigen-binding fragment thereof of as provided herein can be a chimeric antibody, for example, with cytokines or receptor ligands that activate immunifactor cells. For example, BAI1 is a phosphatidylserine recognition receptor that activates phagocytosis (Penberthy, immunological reviews 268: 44-59, 2016).

In certain embodiments, an antibody or fragment thereof of as provided herein can comprise a heavy chain constant region or fragment thereof. For example, the heavy chain can be a murine constant region or fragment thereof or a human constant region or fragment thereof, e.g., IgM, IgG, IgA, IgE, IgD, or IgY constant region or fragment thereof. Various human IgG constant region subtypes or fragments thereof are also contemplated, e.g., a human IgG1, IgG2, IgG3, or IgG4 constant region or fragment thereof. In certain embodiments, the IgG constant region or fragment thereof is a human IgG1 constant region or fragment thereof.

In certain embodiments, an antibody or fragment thereof as provided herein can comprise a light chain constant region or fragment thereof. For example, the light chain constant region or fragment thereof can be a murine constant region or fragment thereof or a human constant region or fragment thereof, e.g., a human kappa or lambda constant region or fragment thereof.

In certain embodiments, the binding domain of an antibody or fragment thereof as provided herein comprises a full-size antibody comprising two heavy chains and two light chains. In other embodiments, the binding domain of an antibody or fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain embodiments, an antibody or antigen-binding fragment thereof as provided herein can be a bispecific antibody or antigen-binding fragment thereof that further comprises a second binding domain. In certain embodiments, the second-binding domain binds to a heterologous antigen or epitope. In certain embodiments, the second binding domain of an antibody or fragment thereof as provided herein comprises a full-size antibody comprising two heavy chains and two light chains. In other embodiments, the second binding domain of an antibody or antigen-binding fragment thereof as provided herein comprises an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain embodiments, an antibody or antigen-binding fragment thereof as provided herein can be conjugated to an anti-cancer agent, a protein, a lipid, a detectable label, a polymer, or any combination thereof.

The disclosure further provides a composition comprising an antibody or antigen-binding fragment thereof as provided for herein, and a carrier. In certain embodiments, the composition comprises an antibody or antigen-binding fragment thereof as provided for herein and a pharmaceutically acceptable carrier and/or excipient.

Also provided for herein is an isolated antibody or antigen-binding fragment thereof comprising a binding domain that specifically binds to Tax interacting protein-1 (TIP1), wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to: SEQ ID NOs: 4, 5, 6, 9, 10, and 11 [v111]: SEQ ID NOs: 15, 16, 17, 12, 13, and 14 [v127]: SEQ ID NOs: 21, 22, 23, 18, 19, and 20 [v154]: or SEQ ID NOs: 27, 28, 29, 24, 25, and 26 [v144], respectively. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for three, two, or one single amino acid substitutions, deletions, or insertions in one or more CDRs to said CDRs. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for two or one single amino acid substitutions, deletions, or insertions in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for one single amino acid substitution, deletion, or insertion in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for four, three, two, or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for three, two, or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for two or one single amino acid substitutions, but not deletions or insertions, in one or more CDRs to said CDR sequences. In certain of any of the aforementioned antibodies or antigen-binding fragment thereof, the binding domain comprises VH-CDR1, VH-CDR2, VH-CDR3, VL-CDR1, VL-CDR2, and VL-CDR3 amino acid sequences identical or identical except for one single amino acid substitution but, not deletion or insertion, in one or more CDRs to said CDR sequences.

### Polynucleotides

Certain embodiments of this disclosure provide for an isolated polynucleotide comprising a nucleic acid encoding an antibody or antigen-binding fragment thereof, or a subunit thereof.

The disclosure further provides a vector comprising a polynucleotide as provided herein and further a composition comprising a polynucleotide or a vector as provided herein.

In certain embodiments the disclosure provides a polynucleotide or a combination of polynucleotides encoding an antibody or antigen-binding fragment thereof.

In certain embodiments of the polynucleotide or combination of polynucleotides as provided herein the nucleic acid encoding a VH and the nucleic acid encoding a VL can be in the same vector. Such a vector is also provided. In certain other embodiments, the polynucleotide or combination of polynucleotides as provided herein comprising the nucleic acid encoding a VH and the nucleic acid encoding a VL can be in different vectors. Such vectors are further provided. The disclosure also provides a host cell comprising the polynucleotide or combination of polynucleotides as provided herein or the vector or vectors as provided.

Moreover, the disclosure provides a method of making an antibody or antigen-binding fragment thereof. Such method comprises culturing a host cell as provided herein and isolating the antibody or antigen-binding fragment thereof.

Provided for herein is an isolated polynucleotide comprising a nucleic acid encoding an antibody or antigen-binding fragment thereof of this disclosure. In certain embodiments, the nucleic acid encodes a VH of said antibody or antigen-binding fragment. In certain embodiments, the nucleic acid encodes a VH, and the VH comprises an amino acid sequence at least 85%, 90%. 95%, 97%, 98%, 99%, or 100% identical to the reference amino acid sequence SEQ ID NO: 3. In certain embodiments, the nucleic acid encodes a VH, and the VH comprises an amino acid sequence 100% identical to SEQ ID NO: 3. In certain embodiments, the nucleic acid comprises the nucleic acid sequence of SEQ ID NO: 2. In certain embodiments, the nucleic acid encodes a VL of said antibody or antigen-binding fragment. In certain embodiments, the nucleic acid encodes a VL, and the VL comprises an amino acid sequence at least 85%, 90%, 95%, 97%, 98%, 99%, or 100% identical to the reference amino acid sequence SEQ ID NO: 8. In certain embodiments, the nucleic acid encodes a VL, and the VL comprises an amino acid sequence 100% identical to SEQ ID NO: 8. In certain embodiments, the nucleic acid comprises the nucleic acid sequence of SEQ ID NO: 7. Certain embodiments provide for a vector comprising any of the aforementioned polynucleotides. Certain embodiments provide for a composition comprising any of the aforementioned polynucleotide or the vectors.

Provided for herein is a polynucleotide or a combination of polynucleotides encoding the antibody or antigen-binding fragment thereof of this disclosure. In certain embodiments, the polynucleotide or combination of polynucleotides comprise a nucleic acid encoding a VH and a nucleic acid encoding a VL. In certain embodiments, the polynucleotide or combination of polynucleotides comprise a nucleic acid encoding a VH and a nucleic acid encoding a VL. In certain embodiments, the VH and VL comprise amino acid sequences at least 85%, 90%, 95%, 97%, 98%, 99%, or 100% identical to reference amino acid sequences SEQ ID NO: 3 and SEQ ID NO: 8, respectively. In certain embodiments, the VH comprises an amino acid sequence at least 85%, 90%, 95%, 97%, 98%, 99%, or 100% identical to reference amino acid sequence SEQ ID NO: 3 and the VL comprises amino acid sequence 100% identical to SEQ ID NO: 8. In certain embodiments, the VH comprises an amino acid sequences 100% identical to SEQ ID NO: 3 and the VL comprises and amino acid sequence at least 85%, 90%, 95%, 97%, 98%, 99%, or 100% identical to reference amino acid sequence and SEQ ID NO: 8. In certain embodiments, the VH and VL comprise amino acid sequences 100% identical to SEQ ID NO: 3 and SEQ ID NO: 8, respectively. In certain embodiments, the nucleic acid encoding the VH and the nucleic acid encoding the VL comprise the nucleic acid sequences of SEQ ID NO: 2 and SEQ ID NO: 7, respectively. In certain embodiments, the nucleic acid encoding a VH and the nucleic acid encoding a VL are in the same vector. Certain embodiments provide for a vector comprising any of the aforementioned polynucleotides or combination of polynucleotides. In certain embodiments, the nucleic acid encoding a VH and the nucleic acid encoding a VL are in different vectors. Certain embodiments provide for vectors comprising any of the aforementioned polynucleotides or combination of polynucleotides.

Also provided for herein is a host cell comprising the polynucleotides, polynucleotide or combination of polynucleotides, or the vector or vectors described above.

Also provided for is a method of making the antibody or antigen-binding fragment of this disclosure, the method comprising: culturing the aforementioned host cell; and isolating the antibody or antigen-binding fragment thereof.

In certain embodiments, the polynucleotides comprise the coding sequence for the mature antibody or antigen-binding fragment thereof, fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) can be used.

Polynucleotide variants are also provided. Polynucleotide variants can contain alterations in the coding regions, non-coding regions, or both. In some embodiments, polynucleotide variants contain alterations that produce silent substitutions, additions, or deletions, but do not alter the properties or activities of the encoded polypeptide. In some embodiments, polynucleotide variants can be produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli). Vectors and cells comprising the polynucleotides described herein are also provided.

In some embodiments, a DNA sequence encoding an antibody or antigen-binding fragment thereof can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest can be inserted into an expression vector and operatively linked to an expression control sequence appropriate for expression of the protein in a desired host. Proper assembly can be confirmed, e.g., by nucleotide sequencing, restriction mapping, and/or expression of a biologically active polypeptide in a suitable host. In order to obtain high expression levels of a transfected gene in a host, the gene can be operatively linked to or associated with transcriptional and translational expression control sequences that are functional in the chosen expression host.

In certain embodiments, recombinant expression vectors can be used to amplify and express DNA encoding an antibody or antigen-binding fragment thereof. Recombinant expression vectors are replicable DNA constructs which have synthetic or cDNA-derived DNA fragments encoding a polypeptide chain of an antibody or and antigen-binding fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which can be transcribed into mRNA and translated into protein, and (3) appropriate transcription and translation initiation and termination sequences, as described in detail below: Such regulatory elements can include an operator sequence to control transcription. The ability to replicate in a host, conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide: a promoter is operatively linked to a coding sequence if it controls the transcription of the sequence: or a ribosome binding site is operatively linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine. This methionine can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

Suitable host cells for expression of an antibody or antigen-binding fragment thereof include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin as described below: Cell-free translation systems could also be employed. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems can also be employed to express an antibody or antigen-binding fragment thereof. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as ribosome binding sites, a polyadenylation site, splice donor and acceptor sites, and transcriptional termination sequences. Baculovirus systems for production of heterologous proteins in insect cells are reviewed by Luckow and Summers, BioTechnology 6:47 (1988).

An antibody or antigen-binding fragment thereof produced by a transformed host, can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify an antibody or antigen-binding fragment thereof. Some or all of the foregoing purification steps, in various combinations, can also be employed to provide a homogeneous recombinant protein.

An antibody or antigen-binding fragment thereof produced in bacterial culture, can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

### Methods of Prevention, Treatment, and Management

Methods are provided for the use of an antibody or antigen-binding fragment thereof of this disclosure, to inhibit binding of TIP1 to TIP1 binding proteins: inhibit cancer cell migration, metastasis, and/or viability; and/or enhances the cytotoxicity of an anti-cancer therapy on a tumor or cancer cell and are considered methods of prevention, treatment, and management in this disclosure.

Methods are provided for the use of an antibody or antigen-binding fragment thereof of this disclosure, to treat patients, for example as a means of prevention, treatment, and management and according for example, to any of the following.

### Pharmaceutical Compositions and Administration Methods

Provided for herein are methods for preventing, treating, and managing cancer in a human subject. In certain embodiments, the method comprises administering to a human in need thereof an effective amount of the antibody or antigen-binding fragment thereof of or the composition of this disclosure. In certain embodiments, the antibody or antigen-binding fragment is co-administered with an anti-cancer therapy. In certain embodiments, the co-administration enhances the cytotoxicity of the anti-cancer therapy on a tumor or cancer cell. And, in certain embodiments, the anti-cancer therapy is radiation therapy.

A single therapeutic antibody can be developed into several different therapeutic agents and thus certain embodiments include antibody-drug conjugates (ADCs), antibody-radioconjugates, bispecific antibodies, fusion antibodies, CART cells, immunotoxins, and other agents. Radioimmunoconjugates (RICs) are another embodiment representing an approach to deliver cytotoxic agents to inducible antigens in cancer. Targeting RICs to radiation-inducible cancer antigens is counterintuitive if merely conjugated to a β-emitter because it would simply add low LET radiation on top of low LET external irradiation. In contrast, conjugating high LET α-emitters or Auger-emitters to antibodies can specifically target very cytotoxic agents to cancer. For example, actinium-225 is an alpha emitter that releases 4 alpha particles per decay with a 10-day half-life. This half-life is well-suited for antibodies with long circulation times (McDevitt M R. Science. 2001:294(5546): 1537-40; Miederer M, Scheinberg D A, McDevitt M R. Advanced Drug Delivery Reviews. 2008:60(12):1371-82). The chelator DTPA was conjugated to anti-TIP1 antibodies and radiolabeled them for imaging and dosimetry (Yan H, et al. Oncotarget. 2016:7(28):43352-62). Cancer selective binding of these RICs was accomplished. Antibodies were labeled with actinium and measured the biodistribution in mouse models to deliver radiopharmaceutics selectively to inducible antigens on lung cancer.

In certain embodiments, antibody drug conjugates (ADCs) are used to deliver cytotoxic drugs specifically to cancer. There are three components to ADCs: antibody, drug, and linker. Linkers are used to conjugate the drug to the antibody. Following endocytosis, the drug dissociates from the antibody and initiates cytotoxicity. This approach has been shown to enhance the efficacy of radiotherapy (Hingorani D V, et al. Molecular cancer therapeutics. 2020:19(1): 157-67; Adams S R, et al. Nature Communications. 2016:7(1):13019). It was observed that the anti-TIP1 antibodies undergo endocytosis following binding on the surface of cancer cells (Lewis C D, et al. Clin Cancer Res. 2021:27(11):3224-33). MMAE chemotherapy was conjugated to anti-TIP1 antibodies to determine whether this radiosensitizing drug can be delivered to irradiated cancers and enhance radiation induced tumor control. These ADCs increased MMAE delivery to cancer and enhanced radiation-induced cytotoxicity and improved tumor control. Thus, it is feasible to deliver radiosensitizing drugs specifically to irradiated cancer and thereby improve bioavailability and reduce systemic toxicities. ADCs are emerging as a rapidly expanding class of therapeutic agents used for cancer treatment. In fact, nine different ADCs are FDA approved for the treatment of multiple cancer types from 2000 until the time of this manuscript and multiple ADCs are in the pipeline for preclinical and clinical development (Khongorzul P, et al. Molecular Cancer Research. 2020; 18(1):3-19; Coats S, et al. Clinical Cancer Research. 2019:25(18):5441-8: Drago J Z, Modi S, Chandarlapaty S. Nature Reviews Clinical Oncology. 2021: Beck A, Goetsch L, Dumontet C, Corvaïa N. Nature Reviews Drug Discovery. 2017: 16(5): 315-37: Verma S, et al. New England Journal of Medicine. 2012:367(19): 1783-91: Lyon R. Drug Discovery Today: Technologies. 2018:30:105-9).

In certain of any of the treatment, prevention, and management embodiments of this disclosure, the antibody or antigen-binding fragment is conjugated to an anti-cancer agent, a protein, a lipid, a detectable label, and/or a polymer, or any combination thereof.

Certain methods provide for detecting a tumor or cancer cell, the method comprising administering to a subject the antibody or antigen-binding fragment thereof, wherein the antibody or antigen-binding fragment is conjugated to a detectable label. Certain embodiments further provide performing a detection step to detect the tumor or cancer cell according to the type of detectable label conjugated to the antibody or antigen-binding fragment, for example radiolabeled for imaging. In certain embodiments, the antibody or antigen-binding fragment can be used to determine patient elligibility for therapy based on level of TIP1 expression in tumor tissue.

Methods of preparing and administering an antibody or antigen-binding fragment thereof provided herein, to a subject in need thereof are well known to or are readily determined by those skilled in the art. The route of administration can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases, a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, an antibody or antigen-binding fragment thereof as provided herein can be delivered directly to a site where the binding molecule can be effective in fighting cancer.

As discussed herein, an antibody or antigen-binding fragment thereof provided herein, can be administered in a pharmaceutically effective amount for the in vivo treatment of diseases or disorders including cancer. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of an antibody or antigen-binding fragment thereof means an amount sufficient to achieve effective binding to a target and to achieve a benefit, e.g., to ameliorate symptoms of a disease or condition or to detect a substance or a cell. Suitable formulations for use in the therapeutic methods disclosed herein can be described in Remington's Pharmaceutical Sciences (Mack Publishing Co.) 16th ed. (1980).

The amount of an antibody or antigen-binding fragment thereof that can be combined with carrier materials to produce a single dosage form will vary depending upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, an antibody or antigen-binding fragment thereof can be administered to a human or other animal in accordance with the aforementioned methods of treatment in an amount sufficient to produce a therapeutic effect. An antibody or antigen-binding fragment thereof provided herein can be administered to such human or other animal in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of an antibody or antigen-binding fragment thereof, that when administered brings about a positive therapeutic response with respect to treatment of a patient with a disease or condition to be treated.

Therapeutically effective doses of the compositions disclosed herein, for the treatment of diseases or disorders including cancer, vary depending upon many different factors, including means of administration, target site, physiological state of the patient, whether the patient is human or an animal, other medications administered, and whether treatment is prophylactic or therapeutic. Usually, the patient is a human, but non-human mammals including non-human primates can also be treated. Treatment dosages can be titrated using routine methods known to those of skill in the art to optimize safety and efficacy.

Factors influencing the mode of administration and the respective amount of an antibody or antigen-binding fragment thereof, but are not limited to, the severity of the disease, the history of the disease, and the age, height, weight, health, and physical condition of the individual undergoing therapy. Similarly, the amount of an antibody or antigen-binding fragment thereof to be administered will be dependent upon the mode of administration and whether the subject will undergo a single dose or multiple doses of this agent.

This disclosure also provides for the use of an antibody or antigen-binding fragment thereof in the manufacture of a medicament for treating, preventing, or managing a disease or disorder such as cancer.

### Kits

This disclosure further provides kits that comprise an antibody or antigen-binding fragment thereof as described herein and that can be used to perform the methods described herein. In certain embodiments, a kit comprises an antibody or antigen-binding fragment thereof, or composition, therapeutic, or diagnostic agents disclosed herein, in one or more containers.

In some embodiments, the kits contain all of the components necessary and/or sufficient to perform a detection assay, including controls, directions for performing assays, and software for analysis and presentation of results.

### Immunoassay's

An antibody or antigen-binding fragment thereof can be assayed for immunospecific binding by any method known in the art. The immunoassay's that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few: Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

In certain embodiments, this disclosure provides a diagnostic kit. In certain embodiments, such a kit comprises a portable immunoassay that can be performed by a healthcare provider at the point-of-care to provide a rapid indication of whether a patient has cancer. In certain embodiments, a diagnostic kit can be used in a method to determine patient eligibility for therapy based on the level of TIP1 expression in tumor tissue.

In certain embodiments, the diagnostic kit provided by the disclosure comprises an antibody or antigen-binding fragment thereof, or a composition comprising such binding molecule as provided herein, and instructions for using the binding molecule or using the composition or directions for obtaining instructions for using the binding molecule or using the composition. In certain embodiments, the kit can be in the form of a test strip, e.g., enclosed in a plastic cassette where the test strip comprises a filter or other solid support. In certain embodiments the binding molecule as provided herein can be associated with the solid support, or can be in a buffer or other solution to be applied to the solid support at some point in the assay. A solid support can be, e.g., a bead, a filter, a membrane or a multiwell plate. In some embodiments, the diagnostic kit is in the form of an enzyme-linked immunosorbent assay (ELISA). For example, the binding molecule as provided herein can be associated with a solid support, a sample obtained from a subject can be applied to the solid support, and any antigen in the subject's sample can be detected with a second antibody. In certain embodiments, the sample can be applied directly to the solid support and can be detected by the binding molecule either elsewhere on the solid support or the antibody can be applied directly to the sample. In each case, the antibody can be detected with a secondary antibody or other reagent conjugated to an enzyme that can be detected by, e.g., a color change.

In certain embodiments, a diagnostic test can be carried out by a carried out at a clinical laboratory using samples provided by a healthcare provider. As used herein, the term “clinical laboratory” refers to a facility for the examination or processing of materials or images derived from a living subject, e.g., a human being. Non-limiting examples of processing include biological, biochemical, serological, chemical, immunohematological, hematological, biophysical, cytological, pathological, genetic, image based, or other examination of materials derived from the human body or of any or all of the human body for the purpose of providing information, e.g., for the diagnosis, prevention, or treatment of any disease or impairment of, or the assessment of the health of living subjects, e.g., human beings. These examinations can also include procedures to collect or otherwise obtain an image, a sample, prepare, determine, measure, or otherwise describe the presence or absence of various substances in the body of a living subject, e.g., a human being, or a sample obtained from the body of a living subject, e.g., a human being.

Using an immunoassay that utilizes an antibody or antigen-binding fragment thereof as provided herein, the user, e.g., a healthcare provider or a clinical laboratory, can determine whether the sample reacts with the antibody or fragment thereof provided in the kit or with an antigen bound to the antibody or fragment thereof (e.g., in a sandwich assay). In certain embodiments the sample can be blood or any fraction thereof, e.g., serum, plasma, or cells, urine, feces, saliva, vomitus, or any combination thereof.

The binding activity of a given lot of an antibody or antigen-binding fragment thereof can be determined according to well-known methods.

Methods and reagents suitable for determination of binding characteristics of an antibody or antigen-binding fragment thereof are known in the art and/or are commercially available. Equipment and software designed for such kinetic analyses are commercially available (e.g., BIAcore®, BIAevaluation® software, GE Healthcare: KINEXA® Software, Sapidyne Instruments).

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al., ed. (1989) Molecular Cloning A Laboratory Manual (2nd ed.: Cold Spring Harbor Laboratory Press); Sambrook et al., ed. (1992) Molecular Cloning: A Laboratory Manual, (Cold Springs Harbor Laboratory, NY): D. N. Glover ed., (1985) DNA Cloning, Volumes I and II: Gait, ed. (1984) Oligonucleotide Synthesis: Mullis et al. U.S. Pat. No. 4,683,195; Hames and Higgins, eds. (1984) Nucleic Acid Hybridization: Hames and Higgins, eds. (1984) Transcription And Translation: Freshney (1987) Culture Of Animal Cells (Alan R. Liss, Inc.): Immobilized Cells And Enzymes (IRL Press) (1986): Perbal (1984) A Practical Guide To Molecular Cloning: the treatise, Methods In Enzymology (Academic Press, Inc., N.Y.): Miller and Calos eds. (1987) Gene Transfer Vectors For Mammalian Cells, (Cold Spring Harbor Laboratory): Wu et al., eds., Methods In Enzymology, Vols. 154 and 155: Mayer and Walker, eds. (1987) Immunochemical Methods In Cell And Molecular Biology (Academic Press, London): Weir and Blackwell, eds., (1986) Handbook Of Experimental Immunology, Volumes I-IV: Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.: Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.: Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York: Stites et al., eds. (1994) Basic and Clinical Immunology (8th ed: Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York: Klein (1982) J., Immunology: The Science of Self-Nonself Discrimination (John Wiley & Sons, NY): Kennett et al., eds. (1980) Monoclonal Antibodies, Hybridoma: A New Dimension in Biological Analyses (Plenum Press, NY): Campbell (1984) “Monoclonal Antibody Technology” in Laboratory Techniques in Biochemistry and Molecular Biology, ed. Burden et al., (Elsevier, Amsterdam): Goldsby et al., eds. (2000) Kuby Immunology (4th ed.: W.H. Freeman & Co.): Roitt et al. (2001) Immunology (6th ed.: London: Mosby): Abbas et al. (2005) Cellular and Molecular Immunology (5th ed.; Elsevier Health Sciences Division): Kontermann and Dubel (2001) Antibody Engineering (Springer Verlag): Sambrook and Russell (2001) Molecular Cloning: A Laboratory Manual (Cold Spring Harbor Press): Lewin (2003) Genes VIII (Prentice Hall, 2003): Harlow and Lane (1988) Antibodies: A Laboratory Manual (Cold Spring Harbor Press): Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### Examples

Bacteriophage-displayed human antibody libraries are used for biopanning. These antibody libraries were created from lymphocyte DNA donated from patients undergoing splenectomy or from peripheral blood lymphocytes. The DNA encoding antibodies is cloned into a phagemid vector and the antibody variable region is expressed on bacteriophage fiber proteins. The resulting antibody library has more than 10⁸ distinct single chain fragments of the variable (scFv) region of human antibodies. Antibodies are selected by binding to the inducible antigens listed above, scFv antibodies are then prioritized when they demonstrate cancer specific binding and high affinity for the inducible antigen. The antibodies are then prioritized based on cancer specific binding and high affinity for the cancer antigen. The antigen binding region (CDR region) of lead antibodies are then grafted into human IgG1. Antibodies are then improved by affinity maturation and reduction of immunogenicity.

Biopanning was performed to identify a human antibody that binds specifically to the PDZ domain of TIP1 through the use of a phage displayed human scFv antibody library. DNA encoding human antibodies were acquired from donors through clinical protocol 2015⁻¹¹⁰¹¹ and a phage library was created using a phagemid vector. Biopanning resulted in the discovery of scFv-154, which showed K^{D} 10⁻⁶ and specificity for TIP1. The fusion antibody, scFv154-Fc showed improved K_{D} to 2×10⁻¹⁰, but a high immunogenicity score >1000.

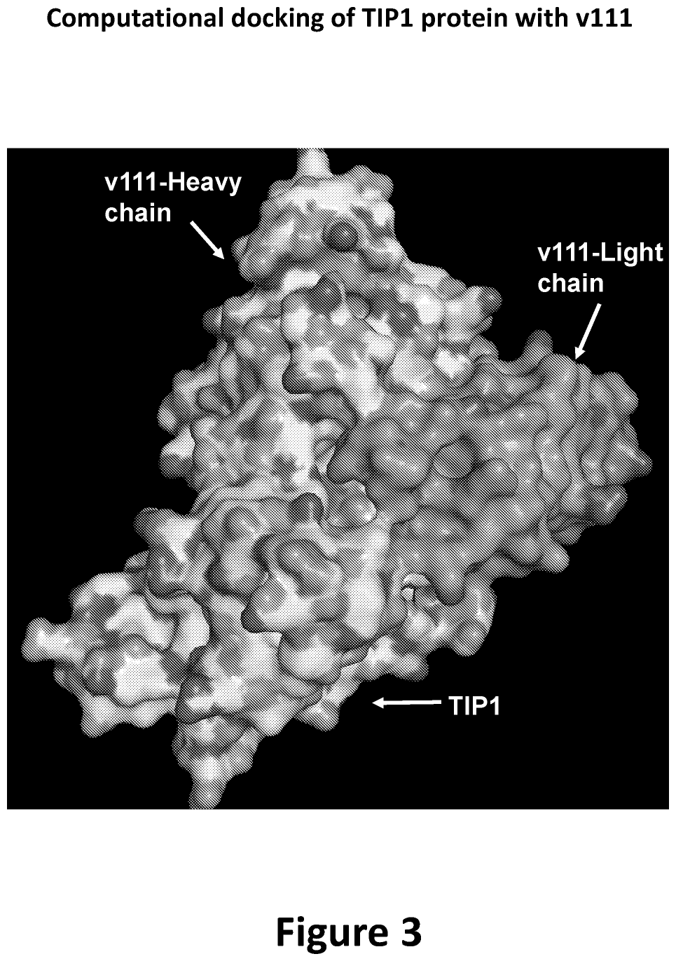

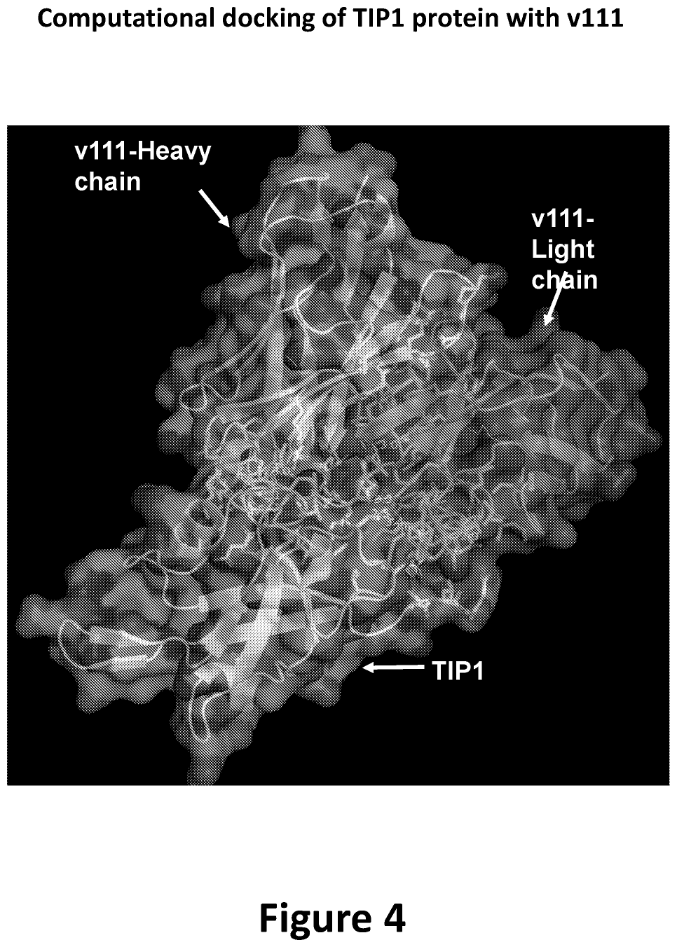

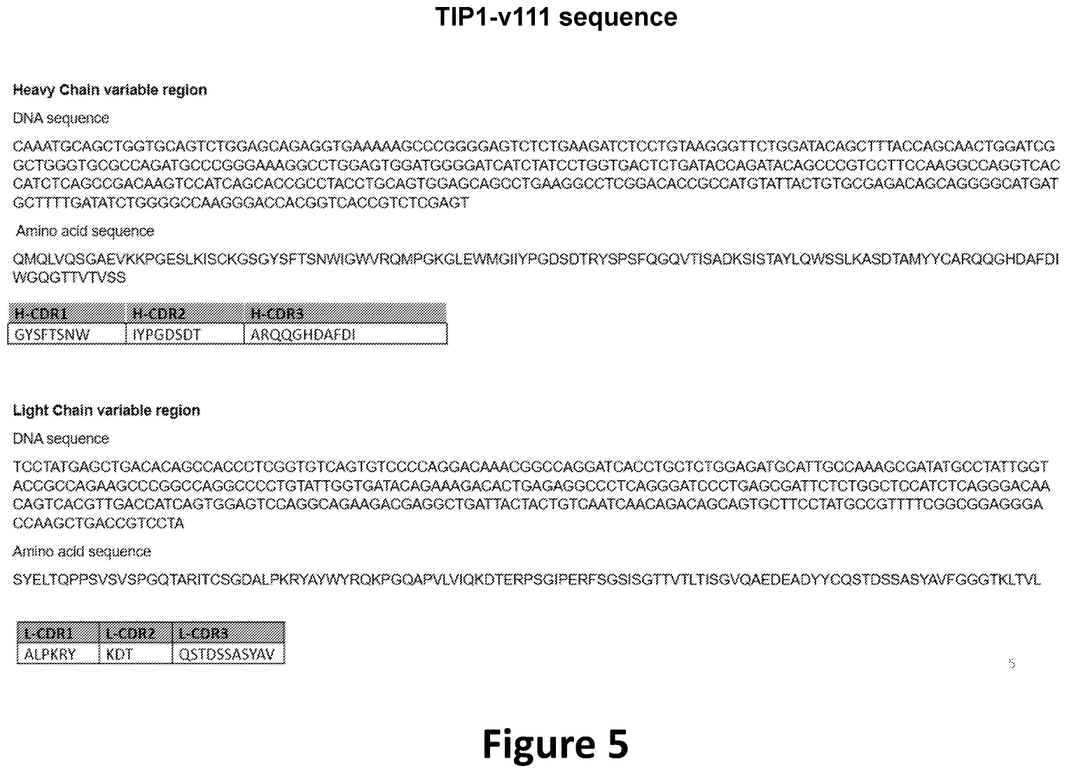

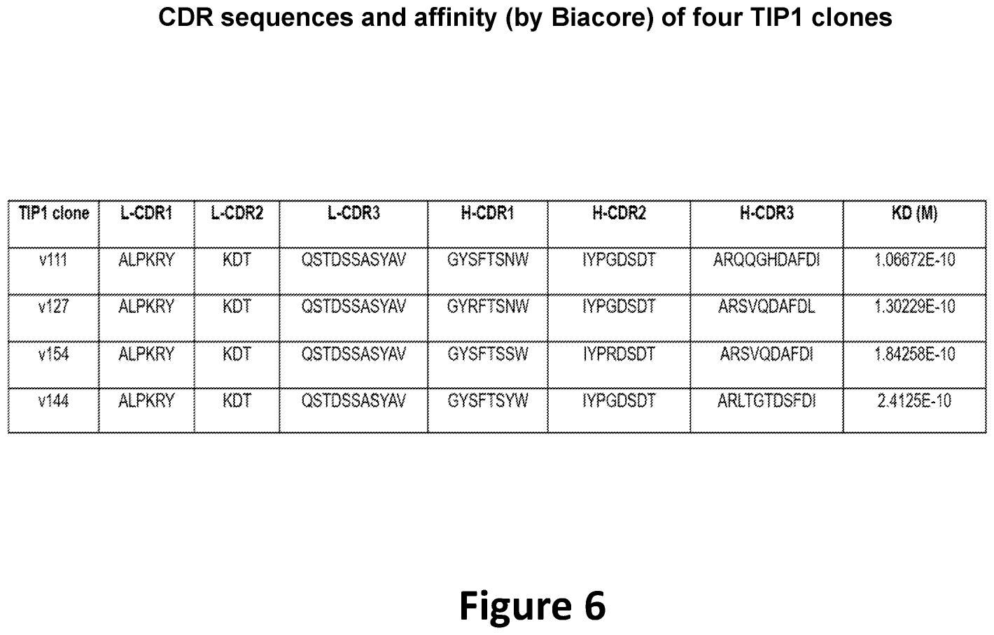

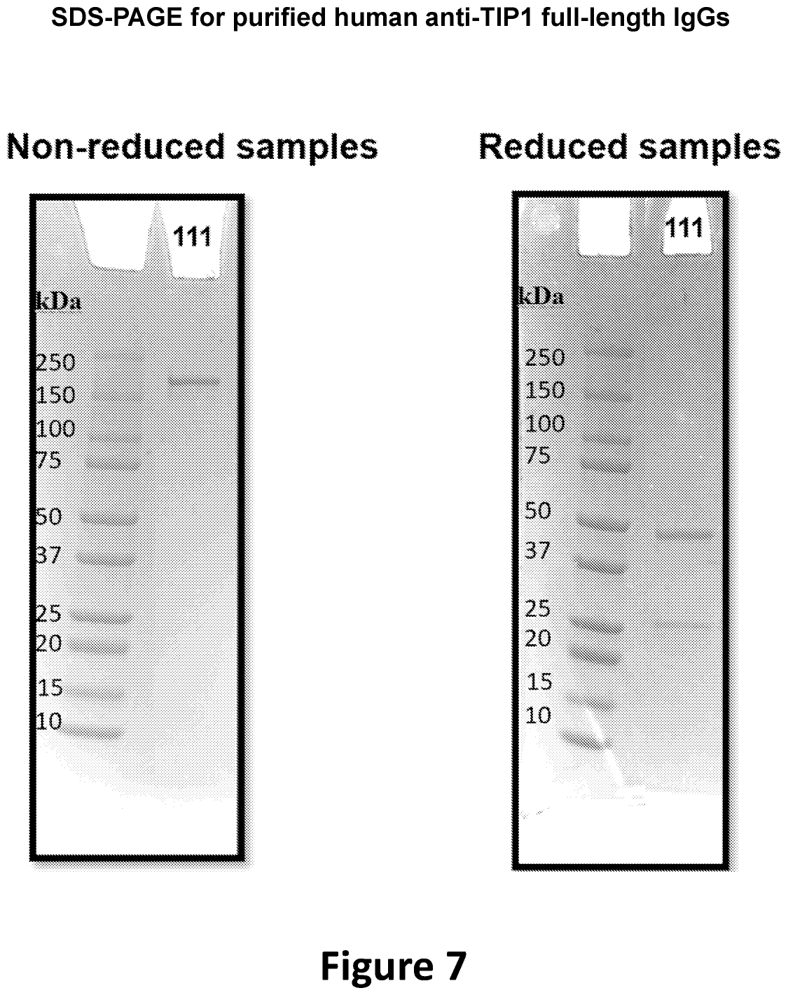

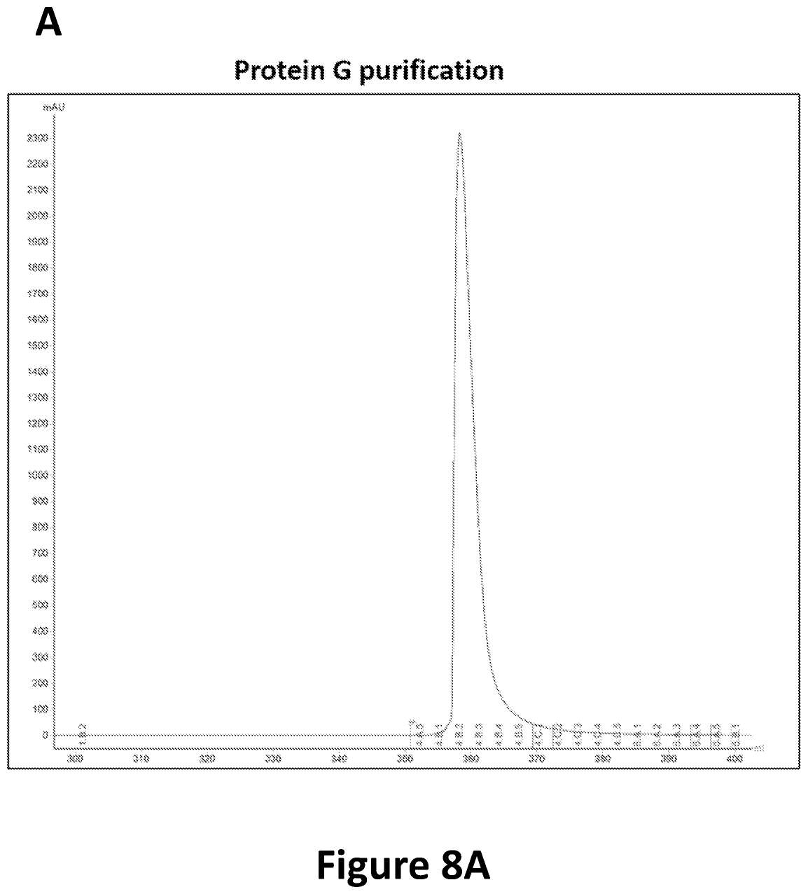

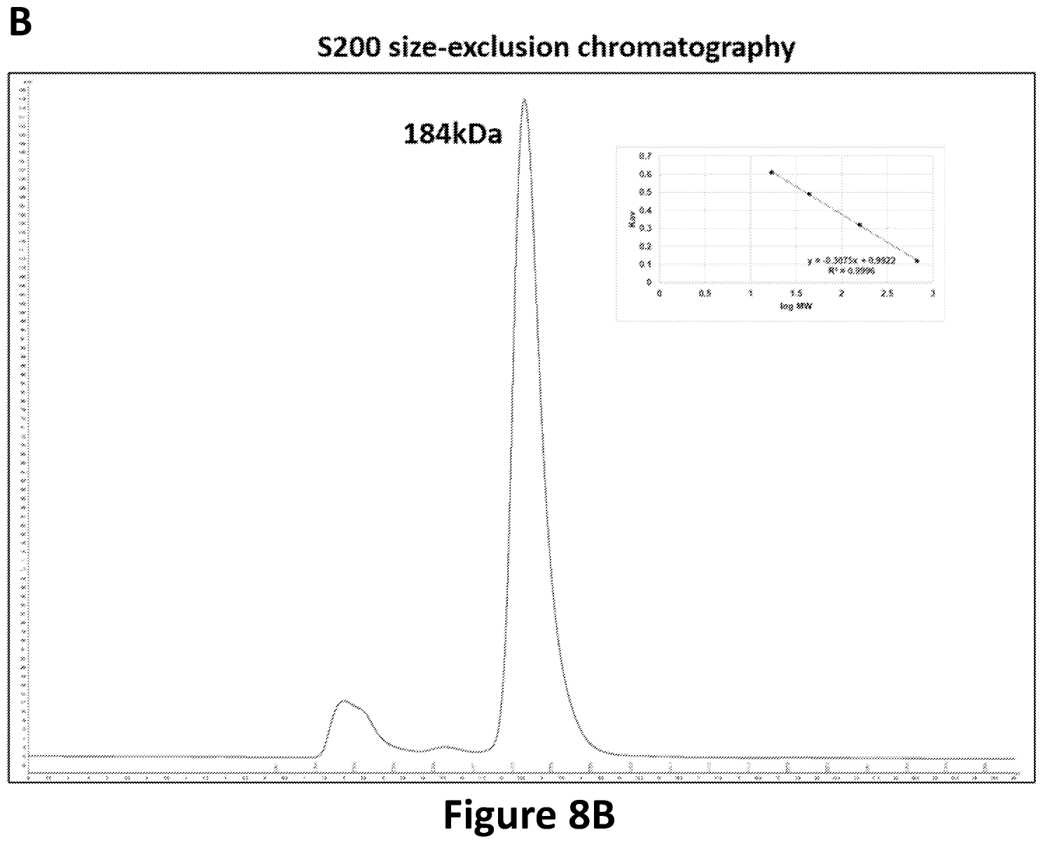

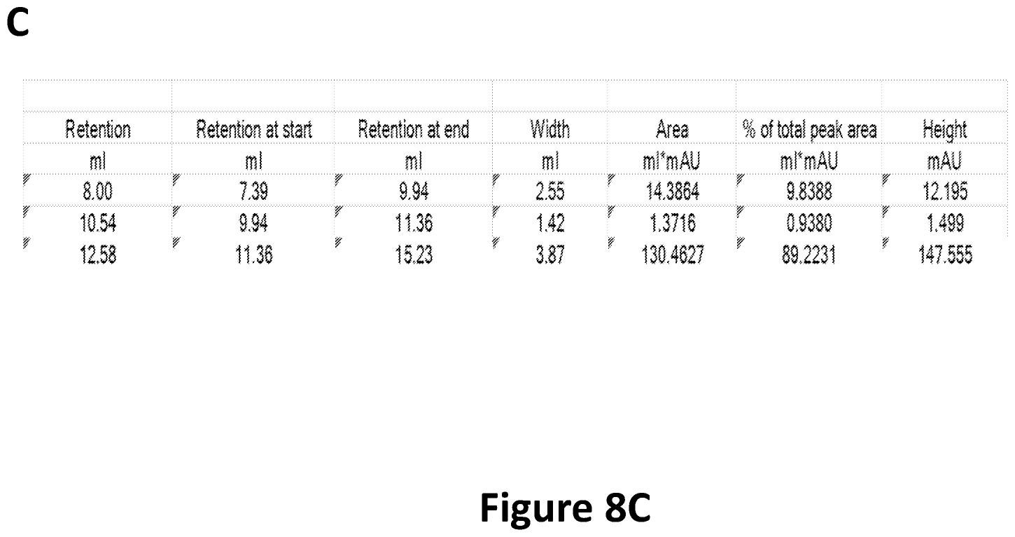

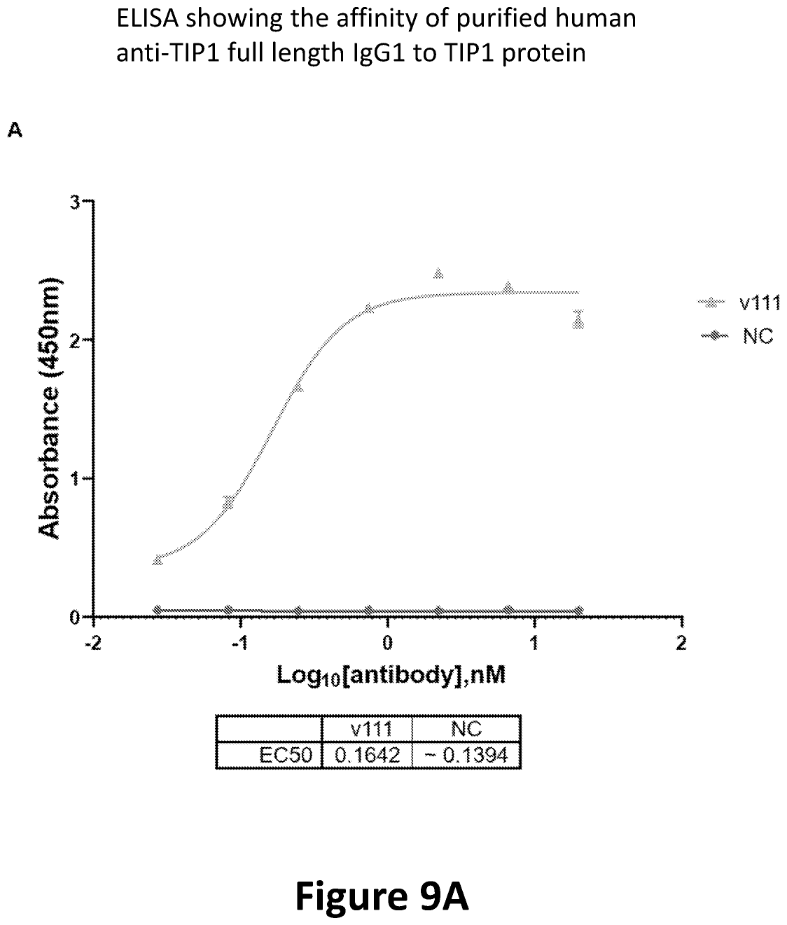

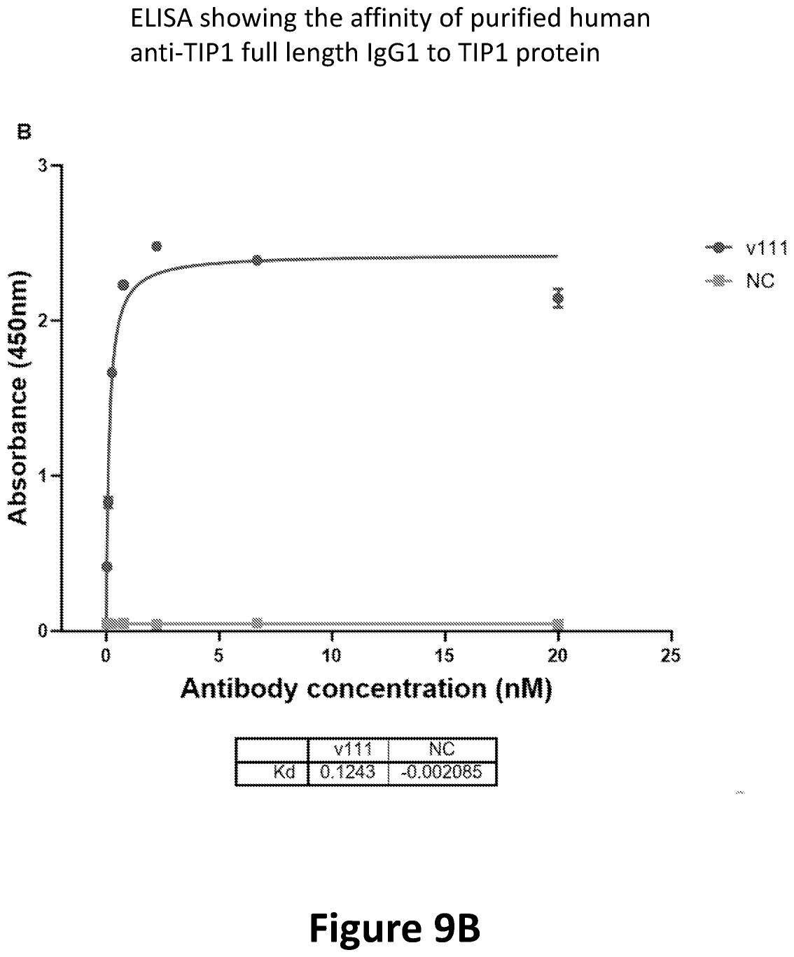

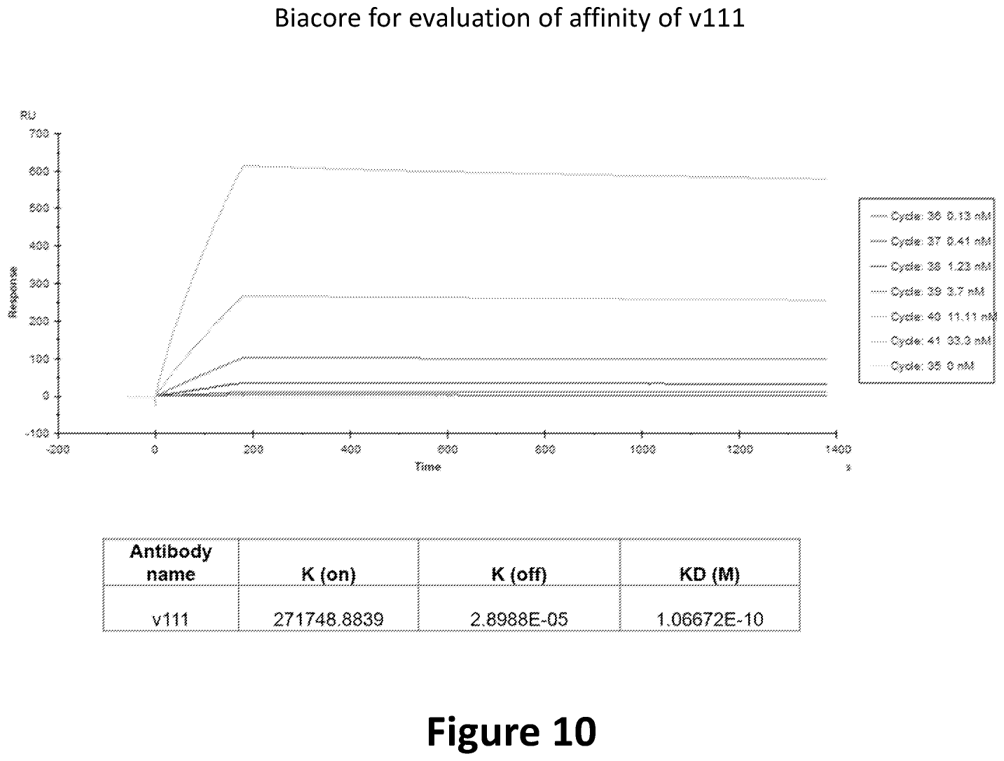

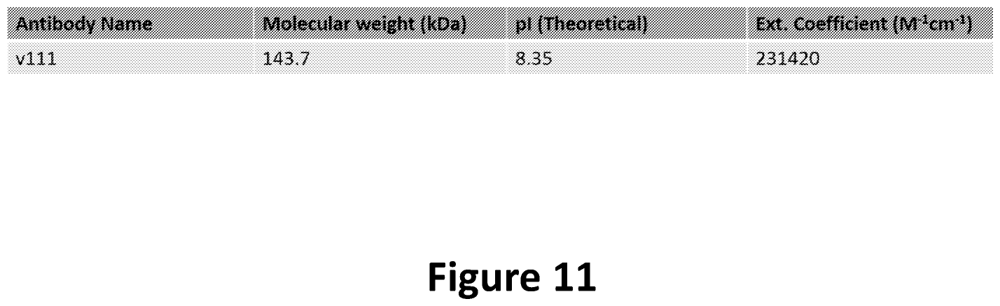

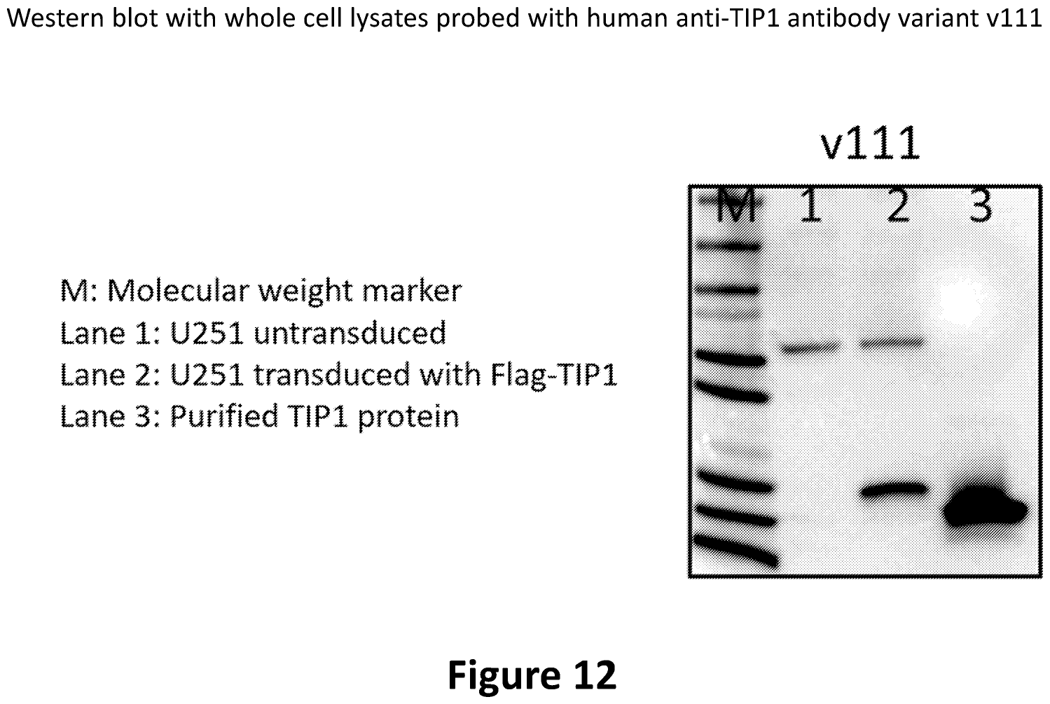

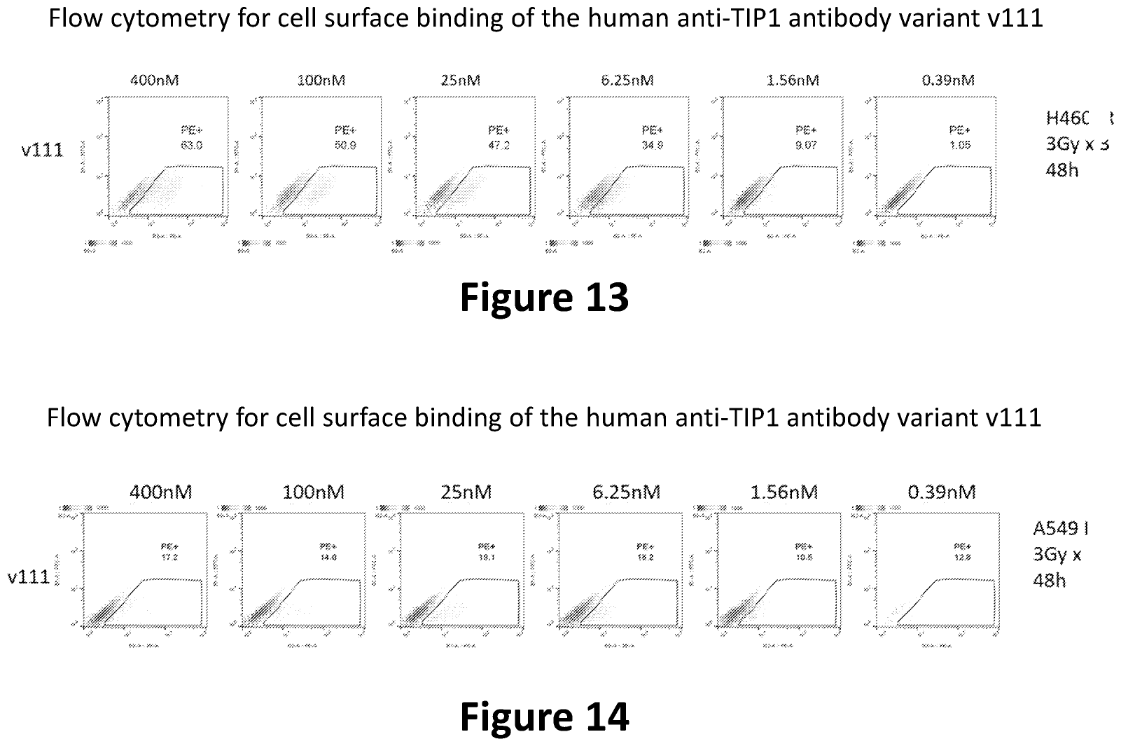

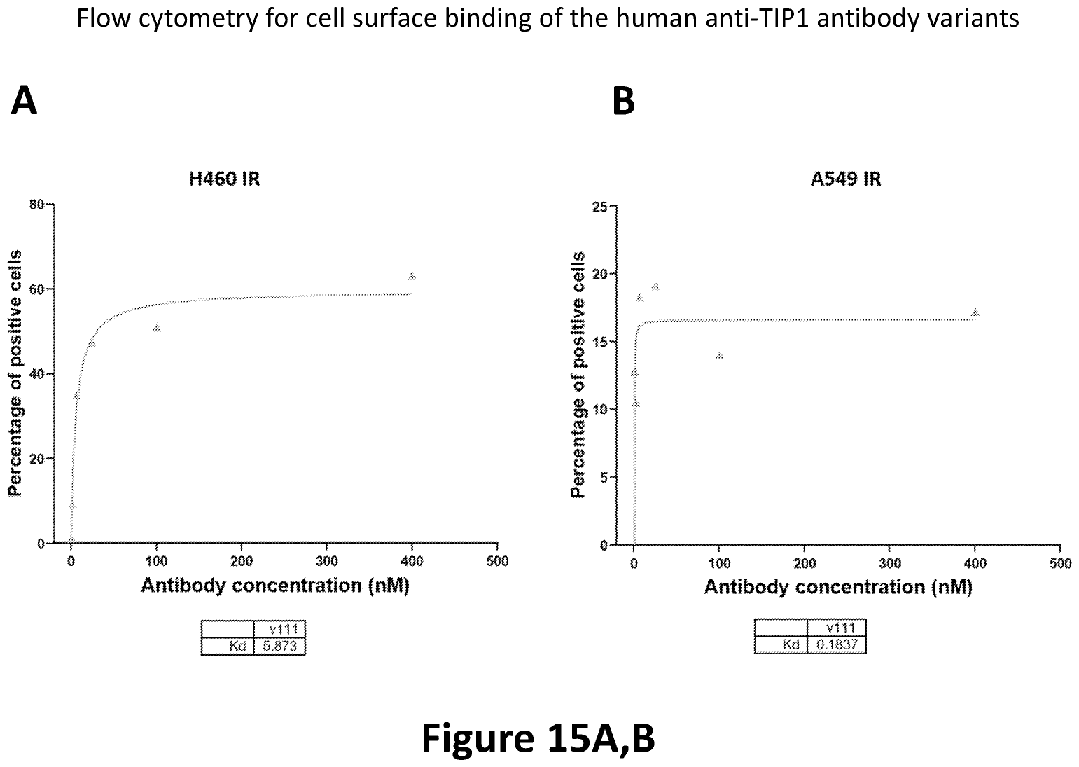

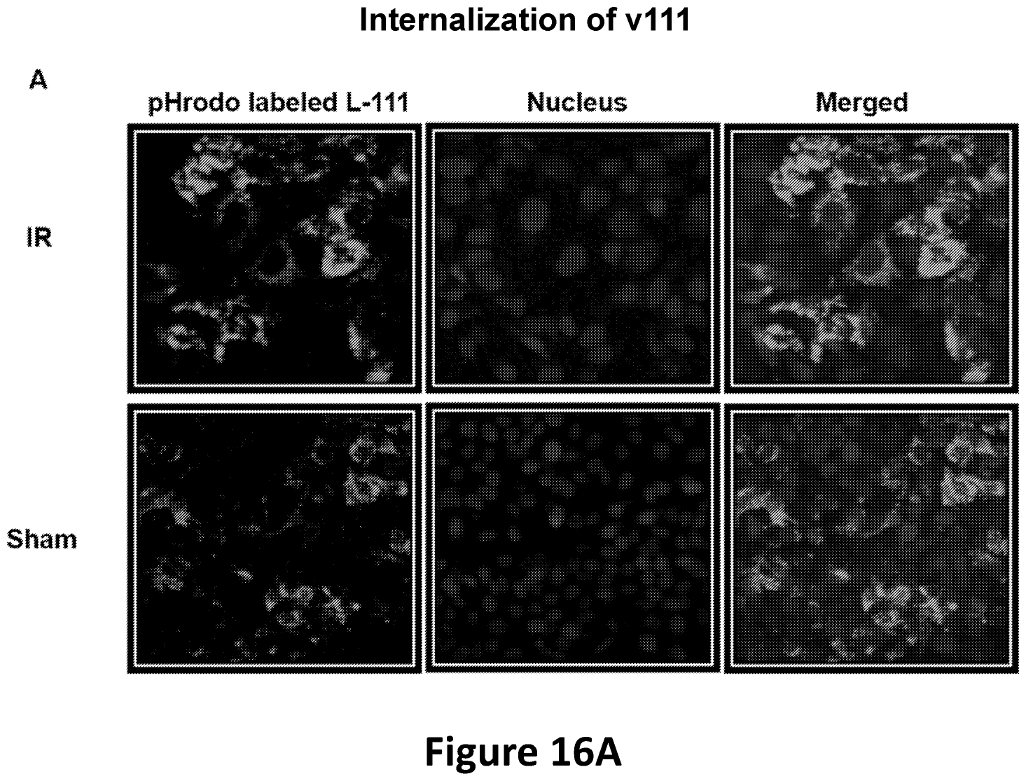

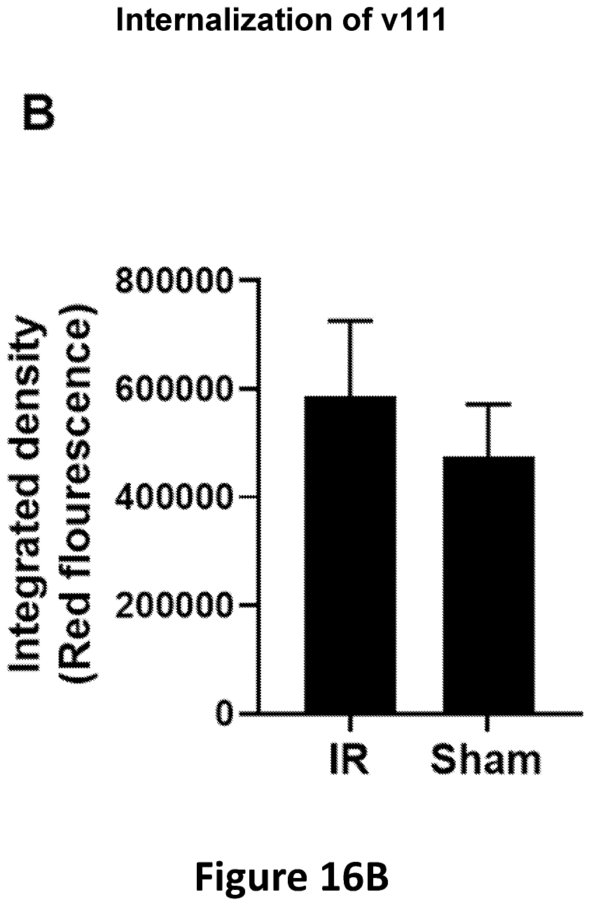

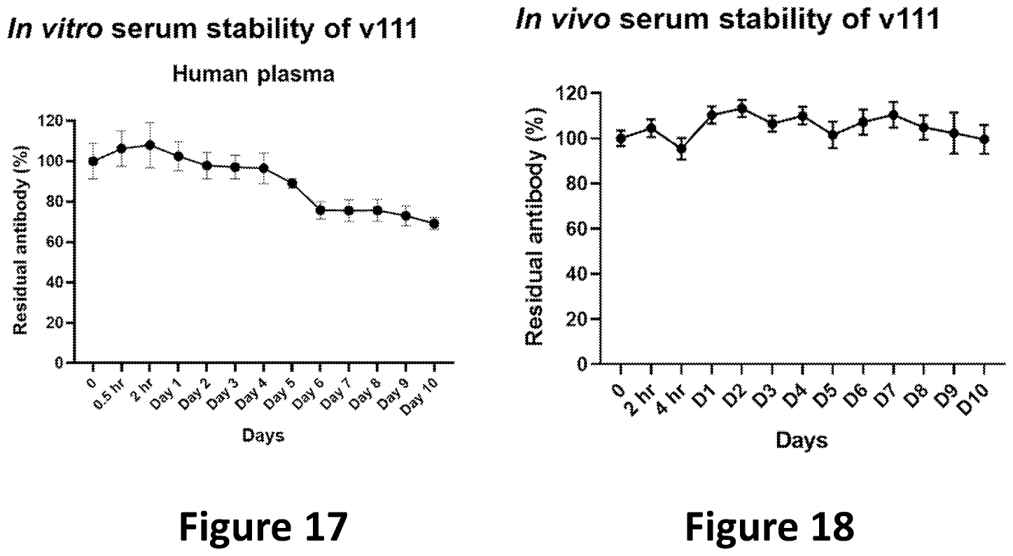

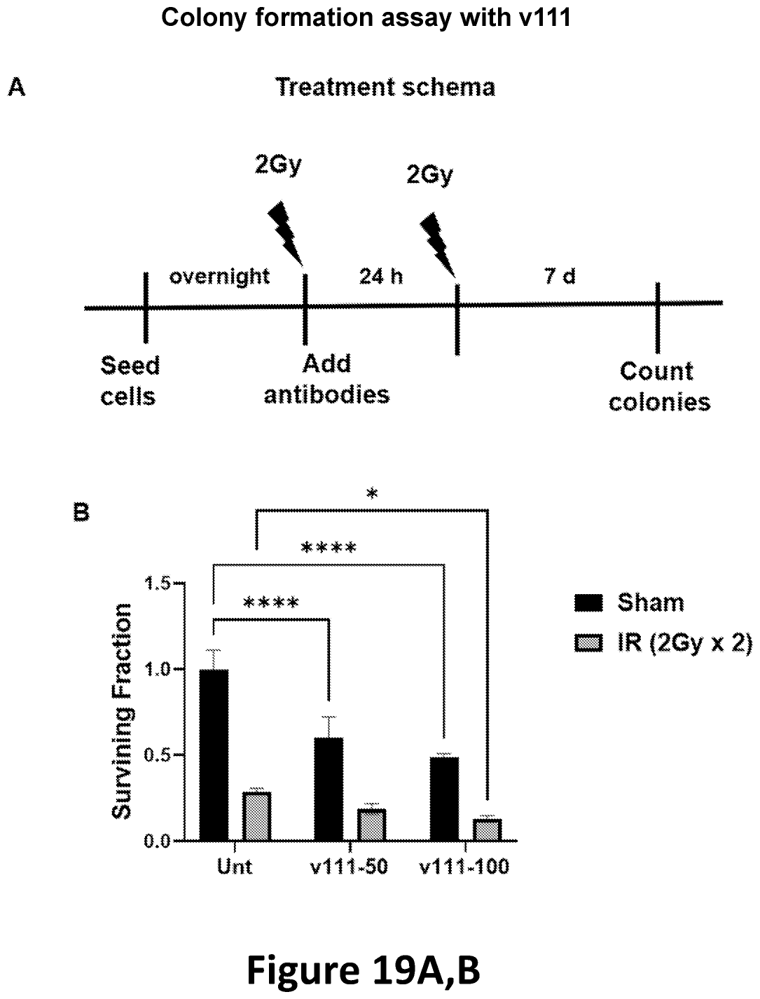

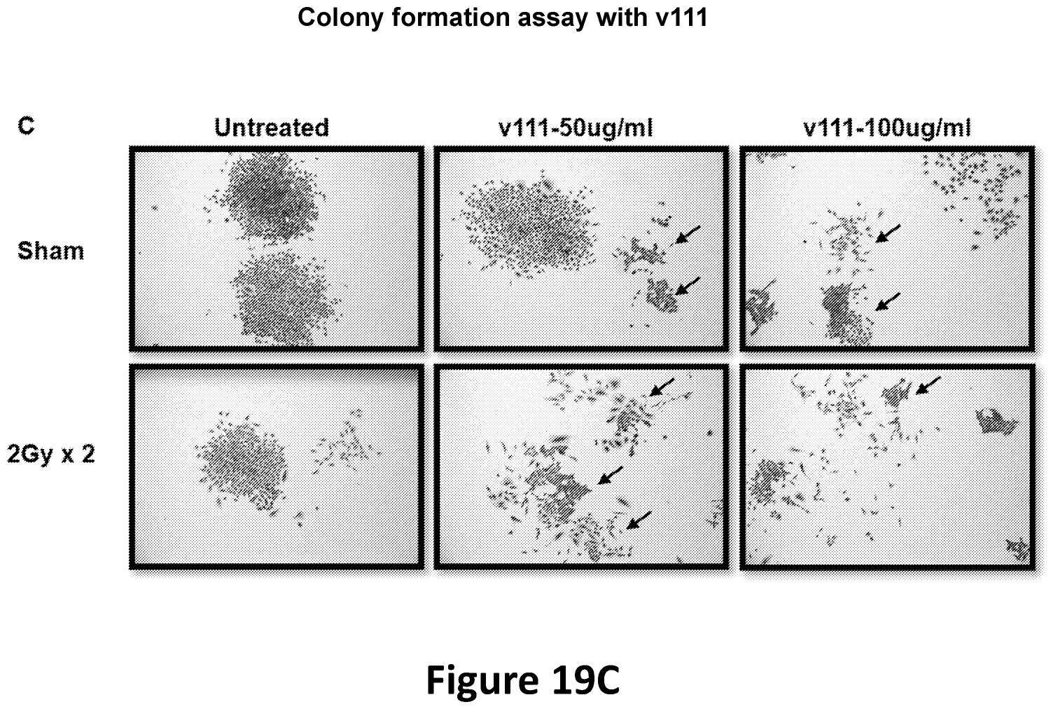

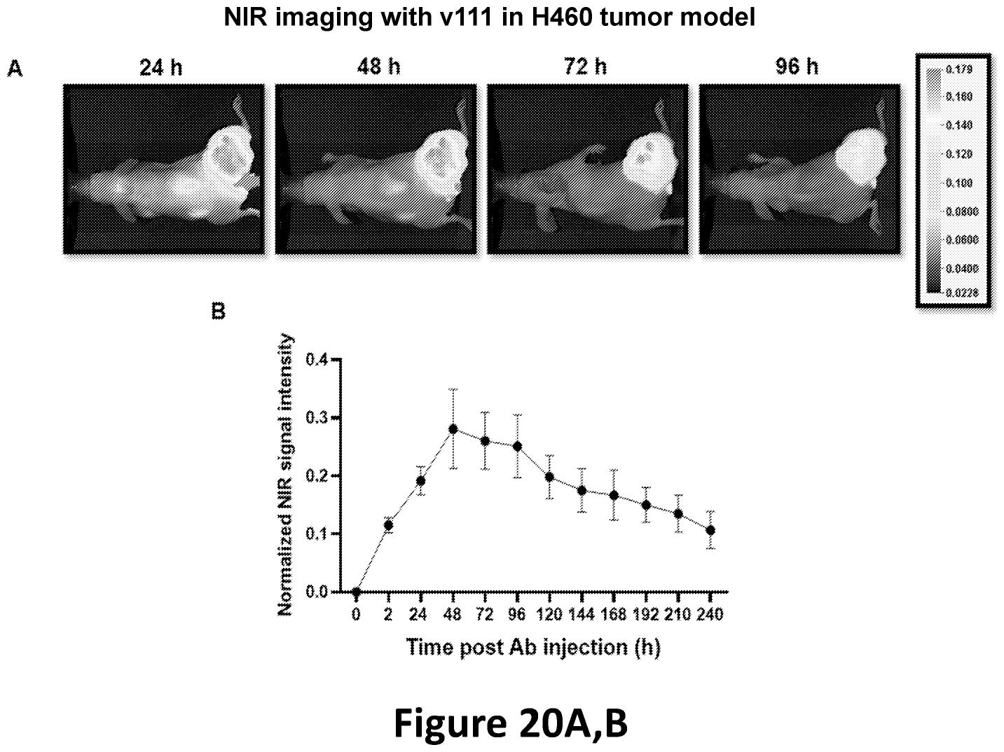

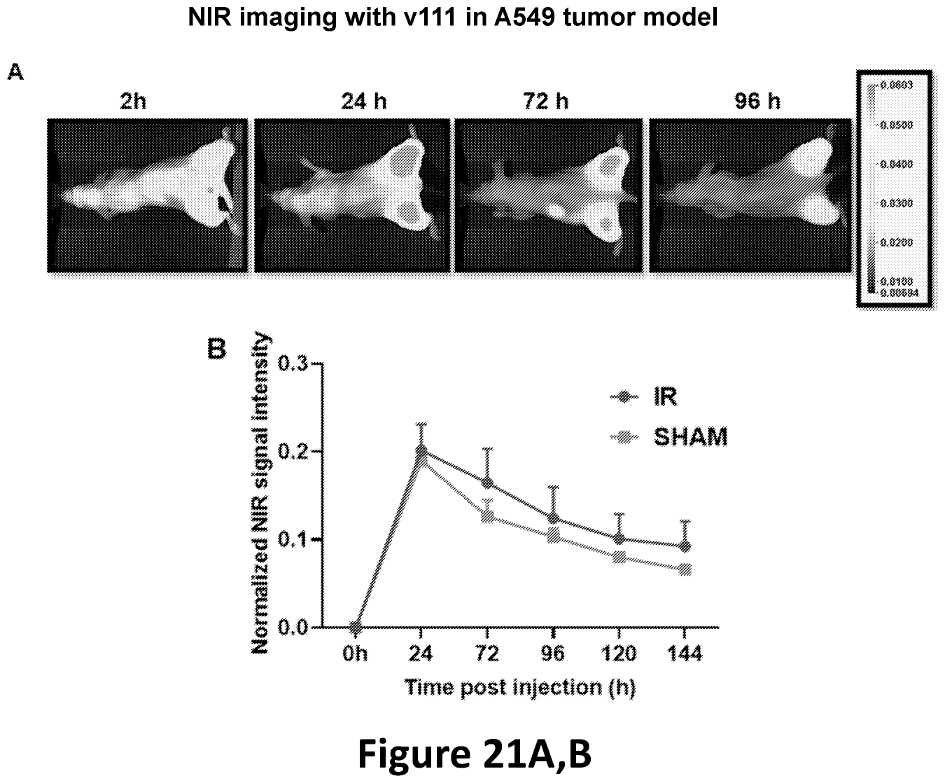

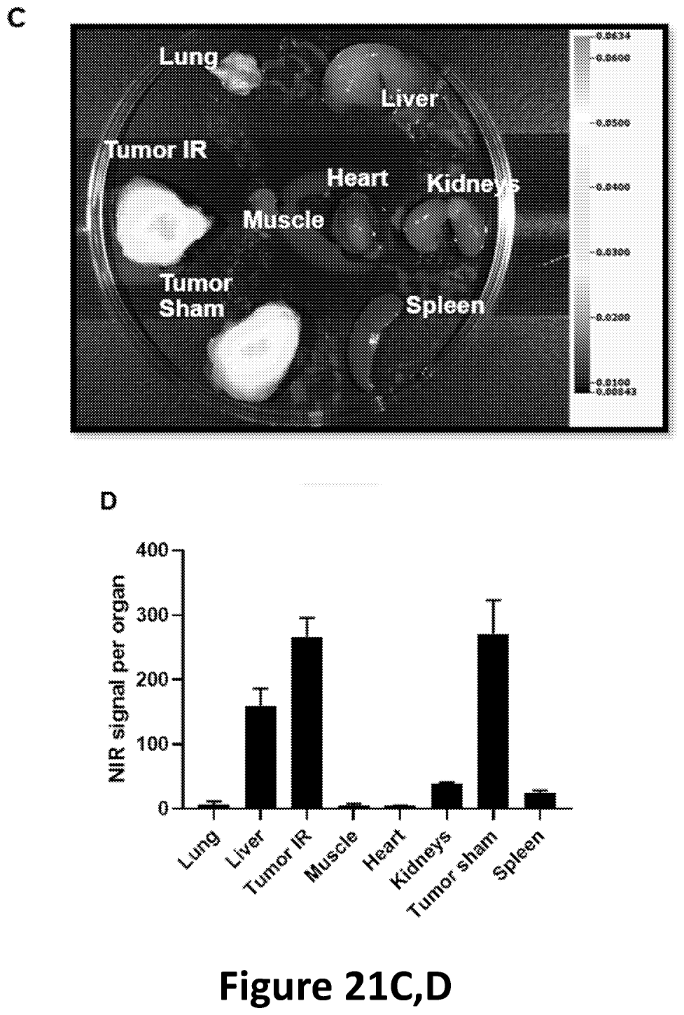

FIG. 2. FIG. 3, and FIG. 4 show computational modeling of scFv-154, which was used to improve the affinity of antibody binding to the PDZ domain of TIP1. Modeling showed that mutations in the heavy chain CDR1 and CDR3 (VH-CDR1 & VH-CDR3) improved affinity for TIP1. FIG. 6 shows the amino acid sequences of L-154 and variants v111, v127, and v144. Certain variations include in CDR1: Aspartic acid replacing Serine (CDR1 S/N) and in CDR3: glutamines replacing serine and valine, and insertion of histidine (CDR3 SVQ/QQGH). The resulting v111 shows improved K_{D} to 8.8×10⁻⁷ as compared to K_{D} 1.8×10⁻⁶ with scFv154.

Immunogenicity was reduced through grafting of the CDRs into the human IgG1 gene. This reduced the immunogenicity score to 600. The human IgG1 v111, resulting from grafting v111 CDRs onto human IgG, showed a K_{D} of 1×10⁻¹⁰ and low immunogenicity score <600.

To characterize the resulting IgG v111, the antibody was run on SDS page under non-reducing and reducing conditions. This shows molecular weight of 150 kDa and heavy and light chains at 50 and 25 kDa (FIG. 7). To determine the affinity of 111, we performed ELISA assay using TIP1 protein (FIG. 9A,B). This shows and affinity that is sub-nanomolar. Similarly. Biacore showed on rate and off rate and K_{D} 10⁻¹⁰ (FIG. 10). The PI is 8.3 and ext, coefficient 231420. FIG. 12 shows IgG v111 immunoblot on total cellular proteins run on Western blot. The blot was probed with IgG v11l. This showed specific binding to TIP1. To determine IgG v111 binding to human cancer cells, human lung cancer cell lines A549, and H460 were studied. This showed antibody binding to cells at sub-nanomolar concentrations (FIG. 13. FIG. 14, and FIG. 15A,B). The antibody was stable in serum (FIG. 17 and FIG. 18).

Antibody delivery of chemotherapy conjugates or radioconjugates is facilitated by endocytosis into cancer cells. The endocytosis of IgG v11l in A549 human lung cancer cells was studied, pHRodo die fluoresces red within the acidic intracellular environment following endocytosis. Microscopy shows rapid endocytosis into live cancer cells (FIG. 16A,B).

It was observed that antibodies that dissociate TIP1 from its binding proteins caused cytotoxicity and enhance efficacy of radiation therapy. Cytotoxicity using human cancer cell line A549 was studied (FIG. 19A). TIP1 was first induced with 2Gy. An antibody was then added at 50 and 100 ug/ml. Cells were irradiated a second time, 24 hours later, in order to measure the interaction between antibody and radiation. FIG. 19B shows cell viability. The antibody enhanced the cytotoxicity of radiation. The antibody alone reduced colony formation (FIG. 19C).

In order to study the cancer specific binding of IgG v111, the antibody was labeled with near infrared die and injected by tail vein into mice bearing irradiated A549 and H460 tumors (FIG. 20A,B and FIG. 21A,B). The results showed that the antibody localized to cancer within 24 hours. The antibody remained bound within the tumor. The right hind limb tumor was treated with radiation and the left hind limb tumor received 0Gy. Antibody IgG v111 also binds to the constitutively expressed TIP1 on the surface of untreated cancer cells, and antibody binding increased in response to irradiation of cancer cells.

Thus it has been demonstrated that IgG v111 is a human IgG1 variant from human scFv-154 with high affinity, cancer specificity, and low immunogenicity and that it activates endocytosis, induces cancer cytotoxicity and enhances radiotherapy.

Certain embodiments of the present disclosure can be defined in any of the following numbered paragraphs:
1. An isolated antibody or antigen-binding fragment thereof comprising a binding domain that specifically binds to a Tax interacting protein-1 (TIP1), wherein the binding domain comprises;
a VH-CDR1 comprising an amino acid sequence identical or identical except for two or one single amino acid substitutions, deletions, or insertions to GYX3FTSX7W (SEQ ID NO: 30), wherein X3 is S or R and X7 is S or N;
a VH-CDR2 comprising an amino acid sequence identical or identical except for two or one single amino acid substitutions, deletions, or insertions to IYPX4DSDT (SEQ ID NO: 31), wherein X4 is G or R;
a VH-CDR3 comprising an amino acid sequence identical or identical except for one single amino acid substitution, deletion, or insertion to ARX3X4X5X6DAFDX12 (SEQ ID NO: 32), wherein X3 is Q or S, X4 is Q or V: X5 is G or Q, X6 is H or absent, and X12 is I or L;
a VL-CDR1 comprising an amino acid sequence identical or identical except for two or one single amino acid substitutions, deletions, or insertions to ALPKRY (SEQ ID NO: 9);
a VL-CDR2 comprising an amino acid sequence identical or identical except for one single amino acid substitutions, deletions, or insertions to KDT (SEQ ID NO: 10); and
a VL-CDR3 comprising an amino acid sequence identical or identical except for three, two, or one single amino acid substitutions, deletions, or insertions to QSTDSSASYAV (SEQ ID NO: 11);
optionally, wherein said TIP1 comprises or consists of SEQ ID NO: 1.
2 The antibody or antigen-binding fragment thereof of paragraph 1, wherein the binding domain comprises:
a VL-CDR1 comprising the amino acid sequence ALPKRY (SEQ ID NO: 9);
a VL-CDR2 comprising the amino acid sequence KDT (SEQ ID NO: 10); and
a VL-CDR3 comprising the amino acid sequence QSTDSSASYAV (SEQ ID NO: 11).
3. The antibody or antigen-binding fragment thereof of paragraph 1 or 2, wherein the binding domain comprises:
a VH-CDR1 comprising the amino acid sequence GYX3FTSX7W (SEQ ID NO: 30), wherein X3 is S or R and X7 is S or N;
a VH-CDR2 comprising the amino acid sequence IYPX4DSDT (SEQ ID NO: 31), wherein X4 is G or R; and
a VH-CDR3 comprising the amino acid sequence ARX3X4X5X6DAFDX12 (SEQ ID NO: 32), wherein X3 is Q or S, X4 is Q or V: X5 is G or Q, X6 is H or absent, and X12 is I or L.
4. The antibody or antigen-binding fragment thereof of paragraph 1 or 2, wherein the binding domain comprises:
a VH-CDR1 comprising the amino acid sequence GYSFTSNW (SEQ ID NO: 4: [v111]) or GYRFTSNW (SEQ ID NO: 15: [v127]);
a VH-CDR2 comprising the amino acid sequence IYPGDSDT (SEQ ID NO: 5); and
a VH-CDR3 comprising the amino acid sequence ARX3X4X5X6DAFDX12 (SEQ ID NO: 32), wherein X3 is Q or S, X4 is Q or V: X5 is G or Q, X6 is H or absent, and X12 is I or L.
5. The antibody or antigen-binding fragment thereof of paragraph 1 or 2, wherein the binding domain comprises:
a VH-CDR1 comprising the amino acid sequence GYSFTSNW (SEQ ID NO: 4: [v111]) or GYRFTSNW (SEQ ID NO: 15: [v127]);
a VH-CDR2 comprising the amino acid sequence IYPGDSDT (SEQ ID NO: 5); and
a VH-CDR3 comprising the amino acid sequence ARQQGHDAFDI (SEQ ID NO: 6; [v111]) or ARSVQDAFDL (SEQ ID NO: 17: [v127]).
6. The antibody or antigen-binding fragment thereof of paragraph 1, wherein the binding domain comprises:
a VH-CDR1 comprising the amino acid sequence GYSFTSNW (SEQ ID NO: 4: [v111]):
a VH-CDR2 comprising the amino acid sequence IYPGDSDT (SEQ ID NO: 5);
a VH-CDR3 comprising the amino acid sequence ARQQGHDAFDI (SEQ ID NO: 6; [v111]);
a VL-CDR1 comprising the amino acid sequence ALPKRY (SEQ ID NO: 9);
a VL-CDR2 comprising the amino acid sequence KDT (SEQ ID NO: 10); and
a VL-CDR3 comprising the amino acid sequence QSTDSSASYAV (SEQ ID NO: 11).
7. The antibody or antigen-binding fragment thereof of paragraph 1, wherein the binding domain comprises:
a VH-CDR1 comprising the amino acid sequence GYRFTSNW (SEQ ID NO: 15; [v127]);
a VH-CDR2 comprising the amino acid sequence IYPGDSDT (SEQ ID NO: 5);
a VH-CDR3 comprising the amino acid sequence ARSVQDAFDL (SEQ ID NO: 17; [v127]);
a VL-CDR1 comprising the amino acid sequence ALPKRY (SEQ ID NO: 9);
a VL-CDR2 comprising the amino acid sequence KDT (SEQ ID NO: 10); and
a VL-CDR3 comprising the amino acid sequence QSTDSSASYAV (SEQ ID NO: 11).
8. The antibody or antigen-binding fragment thereof of any one of paragraphs 1 to 7 comprising said VH-CDR1, VH-CDR2, VH-CDR3, VL-CDR1, VL-CDR2, and VL-CDR3 amino acid sequences, wherein the binding domain comprises VH and VL amino acid sequences at least 85%, 90%, 95%, 97%, 98%, 99%, or 100% identical to reference amino acid sequences SEQ ID NO: 3 [v111] and SEQ ID NO: 8 [v111], respectively.
9 The antibody or antigen-binding fragment thereof of any one of paragraphs 1 to 8, wherein the antibody or antigen-binding fragment specifically binds to TIP1 with a K_{D} value of 1×10⁻⁷ or lower, 9×10⁻⁷ or lower, 8.8×10⁻⁷ or lower, 1×10⁻⁸ or lower, 1×10⁻⁹ or lower, and/or 1×10⁻¹⁰ or lower.
10. The antibody or antigen-binding fragment thereof of any one of paragraphs 1 to 9, wherein the antibody or antigen-binding fragment has an immunogenicity score of less than 1000, less than 900, less than 800, less than 700, less than 600, and/or less than 500.
11. The antibody or antigen-binding fragment thereof of any one of paragraphs 1 to 10, wherein the binding domain specifically binds to the PDZ domain of TIP1.
12. The antibody or antigen-binding fragment thereof of any one of paragraph s 1 to 11, wherein the antibody or antigen-binding fragment can inhibit binding of TIP1 to TIP1 binding proteins.
13. The antibody or antigen-binding fragment thereof of any one of paragraphs 1 to 12, which is a humanized antibody, a chimeric antibody, or antigen-binding fragment thereof.
14. The antibody or antigen-binding fragment thereof any one of paragraphs 1 to 12, which is a humanized antibody or antigen-binding fragment thereof.
15. The antibody or antigen-binding fragment thereof any one of paragraphs 1 to 14, which is a monoclonal antibody, a component of a polyclonal antibody mixture, a recombinant antibody, a multi-specific antibody, or any combination thereof.
16. The antibody or antigen-binding fragment thereof of any one of paragraphs 1 to 15, which is a monoclonal antibody.
17. The antibody or antigen-binding fragment thereof of any one of paragraphs 1 to 16, which comprises a heavy chain constant region or fragment thereof.
18. The antibody or antigen-binding fragment thereof of paragraph 17, wherein the heavy chain constant region or fragment thereof is a human constant region or fragment thereof.
19. The antibody or antigen-binding fragment thereof of paragraph 18, wherein the human heavy chain constant region or fragment thereof is an IgM, IgG, IgA, IgE, IgD, or IgY constant region or fragment thereof.
20. The antibody or antigen-binding fragment thereof of paragraph 19, wherein the human heavy chain constant region or fragment thereof is an IgG constant region or fragment thereof;
optionally wherein the IgG constant region or fragment thereof is a human IgG1, IgG2, IgG3, or IgG4 constant region or fragment thereof.
21. The antibody or antigen-binding fragment thereof of paragraph 20, wherein the IgG constant region or fragment thereof is a human IgG1 constant region or fragment thereof.
22. The antibody or antigen-binding fragment thereof of any one of paragraphs 1 to 21, which comprises a light chain constant region or fragment thereof.
23. The antibody or antigen-binding fragment thereof of paragraph 22, wherein the light chain constant region or fragment thereof is a human constant region or fragment thereof.
24. The antibody or antigen-binding fragment thereof of paragraph 23, wherein the light chain constant region or fragment thereof is human kappa or lambda constant region or fragment thereof.
25. The antibody of any one of paragraph s 1 to 24, comprising a full-size antibody comprising two heavy chains and two light chains.
26. The antibody or antigen-binding fragment thereof of any one of paragraphs 1 to 24, comprising an Fv fragment, an Fab fragment, an F(ab′)2 fragment, an Fab′ fragment, a dsFv fragment, an scFv fragment, an scFab fragment, an sc(Fv)2 fragment, or any combination thereof.
27. The antibody or antigen-binding fragment thereof of any one of paragraphs 1 to 26, wherein the antibody or antigen-binding fragment is bispecific and further comprises a second-binding domain.
28. The antibody or antigen-binding fragment thereof of paragraph 27, wherein the second binding domain comprises a full-size antibody comprising two heavy chains and two light chains or comprises an Fv fragment, an Fab fragment, an F(ab′)2 fragment, an Fab′ fragment, a dsFv fragment, an scFv fragment, an scFab fragment, an sc(Fv)2 fragment, or any combination thereof.
29. The antibody or antigen-binding fragment thereof of any one of paragraphs 1 to 28, wherein binding of the binding domain to TIP1 inhibits cancer cell migration, metastasis, and/or viability.
30. The antibody or antigen-binding fragment thereof of any one of paragraphs 1 to 28, wherein binding of the binding domain to TIP1 enhances the cytotoxicity of an anti-cancer therapy on a tumor or cancer cell.
31. The antibody or antigen-binding fragment thereof of any one of paragraphs 1 to 30, which is conjugated to an anti-cancer agent, a protein, a lipid, a detectable label, and/or a polymer, or any combination thereof.
32. A composition comprising the antibody or antigen-binding fragment thereof of any one of paragraphs 1 to 31, and a pharmaceutically acceptable carrier and/or excipient.
33. A kit, comprising:
   (a) the antibody or antigen-binding fragment thereof of any one of paragraphs 1 to 31 or the composition of paragraph 32; and
   (b) instructions for using the antibody or antigen-binding fragment thereof or using the composition or directions for obtaining instructions for using the antibody or antigen-binding fragment thereof or using the composition.
34. An isolated polynucleotide comprising a nucleic acid encoding the antibody or antigen-binding fragment thereof of any one of paragraphs 1 to 31 or a subunit thereof.
35. The isolated polynucleotide of paragraph 34, wherein the nucleic acid encodes a VH of said antibody or antigen-binding fragment.
36. The polynucleotide of paragraph 34 or 35, wherein the nucleic acid encodes a VH, and wherein the VH comprises an amino acid sequence at least 85%, 90%, 95%, 97%, 98%, 99%, or 100% identical to the reference amino acid sequence SEQ ID NO: 3;
optionally, wherein the nucleic acid comprises the nucleic acid sequence of SEQ ID NO: 2.
37. The polynucleotide of paragraph 34, wherein the nucleic acid encodes a VL of said antibody or antigen-binding fragment.
38. The polynucleotide of paragraph 34 or 37, wherein the nucleic acid encodes a VL, and wherein the VL comprises an amino acid sequence at least 85%, 90%, 95%, 97%, 98%, 99%, or 100% identical to the reference amino acid sequence SEQ ID NO: 8;
optionally, wherein the nucleic acid comprises the nucleic acid sequence of SEQ ID NO: 7.
39. A vector comprising the polynucleotide of any one of paragraphs 34 to 38.
40. A composition comprising the polynucleotide of any one of paragraphs 34 to 38 or the vector of paragraph 39.
41. A polynucleotide or a combination of polynucleotides encoding the antibody or antigen-binding fragment thereof of any one of paragraphs 1 to 31.
42. The polynucleotide or combination of polynucleotides of paragraph 41, comprising a nucleic acid encoding a VH and a nucleic acid encoding a VL.
43. The polynucleotide or combination of polynucleotides of paragraph 41 or paragraph 42, comprising a nucleic acid encoding a VH and a nucleic acid encoding a VL, wherein the VH and VL comprise amino acid sequences at least 85%, 90%, 95%, 97%, 98%, 99%, or 100% identical to reference amino acid sequences SEQ ID NO: 3 and SEQ ID NO: 8, respectively,
optionally, wherein the nucleic acid encoding the VH and the nucleic acid encoding the VL comprise the nucleic acid sequences of SEQ ID NO: 2 and SEQ ID NO: 7, respectively.
44. The polynucleotide or combination of polynucleotides of any one of paragraphs 41 to 43, wherein the nucleic acid encoding a VH and the nucleic acid encoding a VL are in the same vector.
45. A vector comprising the polynucleotide or combination of polynucleotides of paragraph 44.
46. The polynucleotide or combination of polynucleotides of any one of paragraphs 41 to 43, wherein the nucleic acid encoding a VH and the nucleic acid encoding a VL are in different vectors.
47. Vectors comprising the polynucleotide or combination of polynucleotides of paragraph 46.
48. A host cell comprising the polynucleotide or combination of polynucleotides of any one of paragraphs 34 to 38 or 41 to 44 or 46 or the vector or vectors of any one of paragraphs 39, 45 or 47.
49. A method of making the antibody or antigen-binding fragment thereof of any one of paragraphs 1 to 31, comprising
   (a) culturing the cell of paragraph 48; and
   (b) isolating the antibody or antigen-binding fragment thereof.
50. A method for treating cancer in a human subject, the method comprising administering to a human in need thereof an effective amount of the antibody or antigen-binding fragment thereof of any one of paragraphs 1 to 31 or the composition of paragraph 32.
51. Use of an effective amount of the antibody or antigen-binding fragment thereof of any one of paragraphs 1 to 31 or the composition of paragraph 32 for treating cancer in a human subject.
52. The method of paragraph 50 or 51, wherein the antibody or antigen-binding fragment is co-administered and/or used with an anti-cancer therapy,
optionally, wherein said co-administration and/or use enhances the cytotoxicity of the anti-cancer therapy on a tumor or cancer cell.
53. The method of paragraph 52, wherein the anti-cancer therapy is radiation therapy.
54. The method of paragraph 50 or 51, wherein the antibody or antigen-binding fragment is conjugated to an anti-cancer agent, a protein, a lipid, a detectable label, and/or a polymer, or any combination thereof.
55. A method of detecting a tumor or cancer cell, the method comprising administering to a subject the antibody or antigen-binding fragment thereof of any one of paragraphs 1 to 31 or the composition of paragraph 32, wherein the antibody or antigen-binding fragment is conjugated to a detectable label,
optionally, performing a detection step to detect the tumor or cancer cell according to the type of detectable label conjugated to the antibody or antigen-binding fragment.
56. Use of the antibody or antigen-binding fragment thereof of any one of paragraphs 1 to 31 or the composition of paragraph 32 to detect a tumor or cancer cell, wherein the antibody or antigen-binding fragment is conjugated to a detectable label,
optionally, performing a detection step to detect the tumor or cancer cell according to the type of detectable label conjugated to the antibody or antigen-binding fragment.
57. An isolated antibody or antigen-binding fragment thereof comprising a binding domain that specifically binds to Tax interacting protein-1 (TIP1), wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 4, 5, 6, 9, 10, and 11 [v111];
SEQ ID NOs: 15, 16, 17, 12, 13, and 14 [v127];
SEQ ID NOs: 21, 22, 23, 18, 19, and 20 [v154]: or
SEQ ID NOs: 27, 28, 29, 24, 25, and 26 [v144],
respectively.

The breadth and scope of the present disclosure should not be limited by any of the above-described exemplary embodiments, but should be defined only in accordance with the following claims and their equivalents.

### REFERENCES

1. Lewis C D. Singh A K. Hsu F F. Thotala D. Hallahan D E. Kapoor V. Targeting a Radiosensitizing Antibody-Drug Conjugate to a Radiation-Inducible Antigen. Clin Cancer Res. 2021:27(11): 3224-33.
2. Gray J E. Villegas A. Daniel D. Vicente D. Murakami S. Hui R. et al. Three-Year Overall Survival with Durvalumab after Chemoradiotherapy in Stage III NSCLC-Update from PACIFIC. Journal of thoracic oncology: official publication of the International Association for the Study of Lung Cancer. 2020:15(2):288-93.
3. Bonner J A. Harari P M. Giralt J. Cohen R B. Jones C U. Sur R K. et al. Radiotherapy plus cetuximab for locoregionally advanced head and neck cancer: 5-year survival data from a phase 3 randomised trial, and relation between cetuximab-induced rash and survival. The Lancet Oncology. 2010:11(1):21-8.
4. Adams S R. Yang H C. Savariar E N. Aguilera J. Crisp J L. Jones K A. et al. Anti-tubulin drugs conjugated to anti-ErbB antibodies selectively radiosensitive. Nature communications. 2016:7:13019.
5. Hingorani D V. Doan M K. Camargo M F. Aguilera J. Song S M. Pizzo D. et al. Precision Chemoradiotherapy for HER2 Tumors Using Antibody Conjugates of an Auristatin Derivative with Reduced Cell Permeability. Molecular cancer therapeutics. 2020:19(1): 157-67.
6. Hallahan D. Geng L. Qu S. Scarfone C. Giorgio T. Donnelly E. et al. Integrin-mediated targeting of drug delivery to irradiated tumor blood vessels. Cancer Cell. 2003:3(1):63-74.
7. Hariri G. Zhang Y. Fu A. Han Z. Brechbiel M. Tantawy M N, et al. Radiation-Guided P-Selectin Antibody Targeted to Lung Cancer. Annals of Biomedical Engineering. 2008:36(5):821-30.
8. Shamay Y. Elkabets M. Li H. Shah J. Brook S. Wang F. et al. P-selectin is a nanotherapeutic delivery target in the tumor microenvironment. Science Translational Medicine. 2016:8(345): 345ra87-ra87.
9. Elfiky A A. Baghdady A M. Ali S A. Ahmed M I. GRP78 targeting: Hitting two birds with a stone. Life Sci. 2020:260:118317.
10. Dadey D Y A. Kapoor V. Khudanyan A. Thotala D. Hallahan D E. PERK Regulates Glioblastoma Sensitivity to ER Stress Although Promoting Radiation Resistance. Mol Cancer Res. 2018:16(10): 1447-53.
11. Dadey D Y A. Kapoor V. Hoye K. Khudanyan A. Collins A. Thotala D. et al. Antibody Targeting GRP78 Enhances the Efficacy of Radiation Therapy in Human Glioblastoma and Non-Small Cell Lung Cancer Cell Lines and Tumor Models. Clin Cancer Res. 2017:23(10): 2556-64.
12. Dadey D Y. Kapoor V. Khudanyan A. Urano F. Kim A H. Thotala D. et al. The ATF6 pathway of the ER stress response contributes to enhanced viability in glioblastoma. Oncotarget. 2016:7(2): 2080-92.
13. Jaboin J J. Han Z. Hallahan D E. Using in vivo biopanning for the development of radiation-guided drug delivery systems. Methods Mol Biol. 2009:542:285-300.
14. Ferraro D J. Bhave S R. Kotipatruni R P. Hunn J C. Wildman S A. Hong C. et al. High-throughput identification of putative receptors for cancer-binding peptides using biopanning and microarray analysis. Integr Biol (Camb). 2013:5(2):342-50.
15. Han Z. Fu A. Wang H. Diaz. R. Geng L. Onishko H. et al. Noninvasive assessment of cancer response to therapy. Nat Med. 2008:14(3): 343-9.
16. Giordano R J. Cardo-Vila M. Lahdenranta J. Pasqualini R. Arap W. Biopanning and rapid analysis of selective interactive ligands. Nat Med. 2001:7(11): 1249-53.
17. Passarella R J. Zhou L. Phillips J G. Wu H. Hallahan D E, Diaz. R. Recombinant peptides as biomarkers for tumor response to molecular targeted therapy. Clin Cancer Res. 2009:15(20):6421-9.
18. Rasche L. Menoret E. Dubljevic V. Menu E. Vanderkerken K. Lapa C. et al. A GRP78-Directed Monoclonal Antibody Recaptures Response in Refractory Multiple Myeloma with Extramedullary Involvement. Clin Cancer Res. 2016:22(17):4341-9.
19. de Ridder G G. Ray R. Pizzo S V. A murine monoclonal antibody directed against the carboxyl-terminal domain of GRP78 suppresses melanoma growth in mice. Melanoma Res. 2012:22(3): 225-35.
20. Liu R. Li X. Gao W. Zhou Y. Wey S. Mitra S K. et al. Monoclonal antibody against cell surface GRP78 as a novel agent in suppressing PI3K/AKT signaling, tumor growth, and metastasis. Clin Cancer Res. 2013: 19(24): 6802-11.
21. Rauschert N. Brandlein S. Holzinger E. Hensel F. Muller-Hermelink H K. Vollmers H P. A new tumor-specific variant of GRP78 as target for antibody-based therapy. Lab Invest. 2008:88(4): 375-86.
22. Wang H. Han M. Whetsell W. Jr., Wang J. Rich J. Hallahan D. et al. Tax-interacting protein 1 coordinates the spatiotemporal activation of Rho GTPases and regulates the infiltrative growth of human glioblastoma. Oncogene. 2014:33(12): 1558-69.
23. Yan H. Kapoor V. Nguyen K. Akers W J. Li H. Scott J. et al. Anti-tax interacting protein-1 (TIP-1) monoclonal antibody targets human cancers. Oncotarget. 2016:7(28):43352-62.
24. Adams S R. Yang H C. Savariar E N. Aguilera J. Crisp J L. Jones K A, et al. Anti-tubulin drugs conjugated to anti-ErbB antibodies selectively radiosensitize. Nature Communications. 2016:7(1): 13019.
25. Khongorzul P. Ling C J. Khan F U. Ihsan A U. Zhang J. Antibody-Drug Conjugates: A Comprehensive Review: Molecular Cancer Research. 2020:18(1):3-19.
26. Coats S. Williams M. Kebble B. Dixit R. Tseng L. Yao N-S. et al. Antibody-Drug Conjugates: Future Directions in Clinical and Translational Strategies to Improve the Therapeutic Index. Clinical Cancer Research. 2019:25(18):5441-8.
27. Drago J Z. Modi S. Chandarlapaty S. Unlocking the potential of antibody-drug conjugates for cancer therapy. Nature Reviews Clinical Oncology. 2021.
28. Beck A. Goetsch L. Dumontet C. Corvaïa N. Strategies and challenges for the next generation of antibody-drug conjugates. Nature Reviews Drug Discovery. 2017: 16(5):315-37.
29. Verma S. Miles D. Gianni L. Krop I E. Welslau M. Baselga J. et al. Trastuzumab Emtansine for HER2-Positive Advanced Breast Cancer. New England Journal of Medicine. 2012:367(19): 1783-91.
30. Lyon R. Drawing lessons from the clinical development of antibody-drug conjugates. Drug Discovery Today: Technologies. 2018:30:105-9.
31. McDevitt M R. Tumor Therapy with Targeted Atomic Nanogenerators. Science. 2001:294(5546): 1537-40.
32. Miederer M. Scheinberg D A. McDevitt M R. Realizing the potential of the Actinium-225 radionuclide generator in targeted alpha particle therapy applications. Advanced Drug Delivery Reviews. 2008:60(12): 1371-82.

1. An isolated antibody or antigen-binding fragment thereof comprising a binding domain that specifically binds to a Tax interacting protein-1 (TIP1), wherein the binding domain specifically binds to the PDZ domain of TIP1, and comprises:
a VH-CDR1 comprising an amino acid sequence identical or identical except for two or one single amino acid substitutions, deletions, or insertions to GYX₃FTSX₇W (SEQ ID NO: 30), wherein X₃ is S or R and X₇ is S or N;
a VH-CDR2 comprising an amino acid sequence identical or identical except for two or one single amino acid substitutions, deletions, or insertions to IYPX₄DSDT (SEQ ID NO: 31), wherein X₄ is G or R;
a VH-CDR3 comprising an amino acid sequence identical or identical except for one single amino acid substitution, deletion, or insertion to ARX₃X₄X₅X₆DAFDX₁₂ (SEQ ID NO: 32), wherein X₃ is Q or S, X₄ is Q or V; X₅ is G or Q, X₆ is H or absent, and X₁₂ is I or L;
a VL-CDR1 comprising an amino acid sequence identical or identical except for two or one single amino acid substitutions, deletions, or insertions to ALPKRY (SEQ ID NO: 9);
a VL-CDR2 comprising an amino acid sequence identical or identical except for one single amino acid substitutions, deletions, or insertions to KDT (SEQ ID NO: 10); and
a VL-CDR3 comprising an amino acid sequence identical or identical except for three, two, or one single amino acid substitutions, deletions, or insertions to QSTDSSASYAV (SEQ ID NO: 11).

2. The antibody or antigen-binding fragment thereof of claim 1, wherein the binding domain comprises:
a VL-CDR1 comprising the amino acid sequence ALPKRY (SEQ ID NO: 9);
a VL-CDR2 comprising the amino acid sequence KDT (SEQ ID NO: 10); and
a VL-CDR3 comprising the amino acid sequence QSTDSSASYAV (SEQ ID NO: 11).

3. The antibody or antigen-binding fragment thereof of claim 1, wherein the binding domain comprises:
a VH-CDR1 comprising the amino acid sequence GYX₃FTSX₇W (SEQ ID NO: 30), wherein X₃ is S or R and X₇ is S or N;
a VH-CDR2 comprising the amino acid sequence IYPX₄DSDT (SEQ ID NO: 31), wherein X₄ is G or R; and
a VH-CDR3 comprising the amino acid sequence ARX₃X₄X₅X₆DAFDX₁₂ (SEQ ID NO: 32), wherein X₃ is Q or S, X₄ is Q or V; X₅ is G or Q, X₆ is H or absent, and X₁₂ is I or L.

4. The antibody or antigen-binding fragment thereof of claim 1, wherein the binding domain comprises:
a VH-CDR1 comprising the amino acid sequence GYSFTSNW (SEQ ID NO: 4; [v111]) or GYRFTSNW (SEQ ID NO: 15; [v127]);
a VH-CDR2 comprising the amino acid sequence IYPGDSDT (SEQ ID NO: 5); and
a VH-CDR3 comprising the amino acid sequence ARX₃X₄X₅X₆DAFDX₁₂ (SEQ ID NO: 32), wherein X₃ is Q or S, X₄ is Q or V; X₅ is G or Q, X₆ is H or absent, and X₁₂ is I or L.

5. The antibody or antigen-binding fragment thereof of claim 1, wherein the binding domain comprises:
a VH-CDR1 comprising the amino acid sequence GYSFTSNW (SEQ ID NO: 4; [v111]) or GYRFTSNW (SEQ ID NO: 15; [v127]);
a VH-CDR2 comprising the amino acid sequence IYPGDSDT (SEQ ID NO: 5); and
a VH-CDR3 comprising the amino acid sequence ARQQGHDAFDI (SEQ ID NO: 6; [v111]) or ARSVQDAFDL (SEQ ID NO: 17; [v127]).

6. The antibody or antigen-binding fragment thereof of claim 1, wherein the binding domain comprises:
a VH-CDR1 comprising the amino acid sequence GYSFTSNW (SEQ ID NO: 4; [v111]);
a VH-CDR2 comprising the amino acid sequence IYPGDSDT (SEQ ID NO: 5);
a VH-CDR3 comprising the amino acid sequence ARQQGHDAFDI (SEQ ID NO: 6; [v111]);
a VL-CDR1 comprising the amino acid sequence ALPKRY (SEQ ID NO: 9);
a VL-CDR2 comprising the amino acid sequence KDT (SEQ ID NO: 10); and
a VL-CDR3 comprising the amino acid sequence QSTDSSASYAV (SEQ ID NO: 11).

7. (canceled)

8. The antibody or antigen-binding fragment thereof of claim 1 comprising said VH-CDR1, VH-CDR2, VH-CDR3, VL-CDR1, VL-CDR2, and VL-CDR3 amino acid sequences, wherein the binding domain comprises VH and VL amino acid sequences at least 85%, 90%, 95%, 97%, 98%, or 99% identical to reference amino acid sequences SEQ ID NO: 3 [v111] and SEQ ID NO: 8 [v111], respectively.

9. The antibody or antigen-binding fragment thereof of claim 1, wherein the binding domain comprises a VH amino acid sequence of SEQ ID NO: 3 and a VL amino acid sequence of SEQ ID NO: 8.

10-11. (canceled)

12. The antibody or antigen-binding fragment thereof of claim 1, wherein the antibody or antigen-binding fragment can inhibit binding of TIP1 to TIP1 binding proteins.

13-15. (canceled)

16. The antibody or antigen-binding fragment thereof of claim 1, which is a monoclonal antibody.

17-30. (canceled)

31. A composition comprising means for specifically binding to the PDZ domain of TIP1 and a pharmaceutically acceptable carrier and/or excipient.

32. A pharmaceutical composition comprising the antibody or antigen-binding fragment thereof of claim 1, and a pharmaceutically acceptable carrier and/or excipient.

33. (canceled)

34. An isolated polynucleotide comprising a nucleic acid encoding the antibody or antigen-binding fragment thereof of claim 1, or a subunit thereof.

35-40. (canceled)

41. An isolated polynucleotide or a combination of isolated polynucleotides encoding the antibody or antigen-binding fragment thereof of claim 1.

42-47. (canceled)

48. A host cell comprising the isolated polynucleotide of claim 34.

49. A method of making an antibody or antigen-binding fragment thereof, the method comprising:
(a) culturing the cell of claim 48; and
(b) isolating the antibody or antigen-binding fragment thereof.

50. A method for treating cancer in a human subject, the method comprising administering to a human in need thereof an effective amount of the antibody or antigen-binding fragment thereof of claim 1.

51. (canceled)

52. The method of claim 50, wherein the antibody or antigen-binding fragment is co-administered and/or used with an anti-cancer therapy.

53-54. (canceled)

55. A method of detecting a tumor or cancer cell, the method comprising administering to a subject the antibody or antigen-binding fragment thereof of claim 1, wherein the antibody or antigen-binding fragment is conjugated to a detectable label, and performing a detection step to detect the tumor or cancer cell according to the type of detectable label conjugated to the antibody or antigen-binding fragment.

56. (canceled)

57. An isolated antibody or antigen-binding fragment thereof comprising a binding domain that specifically binds to Tax interacting protein-1 (TIP1), wherein the binding domain comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to:
SEQ ID NOs: 4, 5, 6, 9, 10, and 11 [v111];
SEQ ID NOs: 15, 16, 17, 12, 13, and 14 [v127];
SEQ ID NOs: 21, 22, 23, 18, 19, and 20 [v154]; or
SEQ ID NOs: 27, 28, 29, 24, 25, and 26 [v144],
respectively.


SEQUENCE LISTING (35 sequences)

SEQ ID NO: 1 - protein, 124 aa, Homo sapiens
MSYIPGQPVTAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQKAVQQSMLS

SEQ ID NO: 2 - DNA, 354 bp, synthetic construct
  source 1..354: mol_type = other DNA
caaatgcagctggtgcagtctggagcagaggtgaaaaagcccggggagtctctgaagatctcctgtaagggttctggatacagctttaccagcaactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagcaggggcatgatgcttttgatatctggggccaagggaccacggtcaccgtctcgagt

SEQ ID NO: 3 - protein, 118 aa, synthetic construct
QMQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQQGHDAFDIWGQGTTVTVSS

SEQ ID NO: 4 - protein, 8 aa, synthetic construct
GYSFTSNW

SEQ ID NO: 5 - protein, 8 aa, synthetic construct
IYPGDSDT

SEQ ID NO: 6 - protein, 11 aa, synthetic construct
ARQQGHDAFDI

SEQ ID NO: 7 - DNA, 324 bp, synthetic construct
  source 1..324: mol_type = other DNA
tcctatgagctgacacagccaccctcggtgtcagtgtccccaggacaaacggccaggatcacctgctgtggagatgcattgccaaagcgatatgcctattggtaccgccagaagcccggccaggcccctgtattggtgatacagaaagacactgagaggccctcagggatccctgagcgattctctggctccatctcagggacaacagtcacgttgaccatcagtggagtccaggcagaagacgaggctgattactactgtcaatcaacagacagcagtgcttcctatgccgttttcggcggagggaccaagctgaccgtccta

SEQ ID NO: 8 - protein, 108 aa, synthetic construct
SYELTQPPSVSVSPGQTARITCSGDALPKRYAYWYRQKPGQAPVLVIQKDTERPSGIPERFSGSISGTTVTLTISGVQAEDEADYYCQSTDSSASYAVFGGGTKLTVL

SEQ ID NO: 9 - protein, 6 aa, synthetic construct
ALPKRY

SEQ ID NO: 10 - skipped

SEQ ID NO: 11 - protein, 11 aa, synthetic construct
QSTDSSASYAV

SEQ ID NO: 12 - protein, 6 aa, synthetic construct
ALPKRY

SEQ ID NO: 13 - skipped

SEQ ID NO: 14 - protein, 11 aa, synthetic construct
QSTDSSASYAV

SEQ ID NO: 15 - protein, 8 aa, synthetic construct
GYRFTSNW

SEQ ID NO: 16 - protein, 8 aa, synthetic construct
IYPGDSDT

SEQ ID NO: 17 - protein, 10 aa, synthetic construct
ARSVQDAFDL

SEQ ID NO: 18 - protein, 6 aa, synthetic construct
ALPKRY

SEQ ID NO: 19 - skipped

SEQ ID NO: 20 - protein, 11 aa, synthetic construct
QSTDSSASYAV

SEQ ID NO: 21 - protein, 8 aa, synthetic construct
GYSFTSSW

SEQ ID NO: 22 - protein, 8 aa, synthetic construct
IYPRDSDT

SEQ ID NO: 23 - protein, 10 aa, synthetic construct
ARSVQDAFDI

SEQ ID NO: 24 - protein, 6 aa, synthetic construct
ALPKRY

SEQ ID NO: 25 - skipped

SEQ ID NO: 26 - protein, 11 aa, synthetic construct
QSTDSSASYAV

SEQ ID NO: 27 - protein, 8 aa, synthetic construct
GYSFTSYW

SEQ ID NO: 28 - protein, 8 aa, synthetic construct
IYPGDSDT

SEQ ID NO: 29 - protein, 11 aa, synthetic construct
ARLTGTDSFDI

SEQ ID NO: 30 - protein, 8 aa, synthetic construct
  VARIANT 3: note = S or R
  VARIANT 7: note = S or N
GYXFTSXW

SEQ ID NO: 31 - protein, 8 aa, synthetic construct
  VARIANT 4: note = G or R
IYPXDSDT

SEQ ID NO: 32 - protein, 11 aa, synthetic construct
  VARIANT 3: note = Q or S
  VARIANT 4: note = Q or V
  VARIANT 5: note = G or Q
  VARIANT 6: note = H or absent
  VARIANT 11: note = I or L
ARXXXXDAFDX

SEQ ID NO: 33 - protein, 6 aa, synthetic construct
HHHHHH

SEQ ID NO: 34 - protein, 4 aa, synthetic construct
QQGH

SEQ ID NO: 35 - protein, 10 aa, Homo sapiens
QNPFSEDKTD